(19) United States
(12) Patent Application Publication
(10) Pub. No.: US 2023/0272101 A1
(43) Pub. Date: Aug. 31, 2023

(54) DENDRITIC CELL ACTIVATING THERAPY AS AN ADJUNCT TO RADIATION THERAPY

(71) Applicant: Montefiore Medical Center, Bronx, NY (US)

(72) Inventors: Chandan GUHA, Scarsdale, NY (US); Sanjay PANDEY, Bronx, NY (US)

(21) Appl. No.: 18/007,356

(22) PCT Filed: Aug. 5, 2021
(86) PCT No.: PCT/US2021/044856

§ 371 Date: Jan. 30, 2023

Related U.S. Application Data
(60) Provisional application No. 63/062,185, filed on Aug. 6, 2020.

(51) Int. Cl. C07K 16/28; A61P 35/00; A61P 37/04; A61N 5/02; A61N 7/00; A61B 18/12; A61N 5/10

(52) U.S. Cl. CPC C07K 16/2878; A61B 18/12; A61N 5/02; A61N 5/1077; A61N 7/00; A61P 35/00; A61P 37/04; A61K 2039/505

(57) ABSTRACT

Provided herein are methods relating to administering a dendritic cell activating therapy as an adjunct to radiation therapy or an energy-based therapy for treating a tumor or cancer in an individual.

### CROSS-REFERENCE

This application claims the benefit of U.S. Provisional Application No. 63/062,185, filed Aug. 6, 2020, which application is incorporated herein by reference herein in its entirety.

### SUMMARY

Radiation therapy is commonly used as a treatment for cancer. Radiation therapy uses ionizing radiation to damage the genetic material of the targeted cells, resulting in death and damage of the affected cells. However, in many circumstances, radiation therapy is not sufficient to eradicate all remnants of a cancer and/or tumor, or prevent distal metastases of a cancer.

Dendritic cells are antigen-presenting cells which process antigenic material and present it on the cell surface to the T-cells of the immune system. When T-cells are presented with tumor specific antigens by dendritic cells, the T-cells are then able to play a critical role in the immune system’s ability to target and kill tumor cells. This disclosure describes uses of dendritic cell activating molecules that improve the effectiveness of radiation treatment, and establish systemic anti-cancer/tumor immunity. In these methods, the dendritic cell activating molecule is administered after radiation treatment. This results in improved treatment of the cancer or tumor compared to treatment with radiation or dendritic cell activating molecule alone, as well as when compared to simultaneous treatment with radiation and a dendritic cell activating molecule.

Described herein in one aspect is a method of treating a tumor or a cancer in an individual, the method comprising administering to the individual a dose of a radiation therapy and a dendritic cell activating molecule, wherein the dendritic cell activating molecule is administered at least one day after the radiation therapy is administered. Also described is a method of treating a tumor or a cancer in an individual, the method comprising administering to the individual a dendritic cell activating molecule, wherein the individual has received a dose of a radiation therapy, and wherein the dendritic cell activating molecule is administered at least one day after the radiation therapy has been administered. In certain embodiments, the dendritic cell activating molecule is administered at least two days after the radiation therapy is administered. In certain embodiments, the dendritic cell activating molecule is administered at least three days after the radiation therapy is administered. In certain embodiments, the dose of the radiation therapy comprises a plurality of doses of radiation therapy. In certain embodiments, the radiation therapy is external beam radiation therapy. In certain embodiments, the external beam radiation therapy is selected from the list consisting of: three-dimensional conformal radiation therapy, intensity modulated radiation therapy, image guided radiation therapy, stereotactic radiation therapy, intraoperative radiation therapy, proton beam therapy, neutron beam therapy, and combinations thereof. In certain embodiments, the dose of radiation therapy comprises at least about 2 Gy. In certain embodiments, the dose of radiation therapy comprises at least about 2 Gy and no more than about 20 Gy. In certain embodiments, the dendritic cell activating molecule is administered at least three days after the dose of the radiation therapy. In certain embodiments, the dendritic cell activating molecule is administered at least five days after the dose of the radiation therapy. In certain embodiments, the dendritic cell activating molecule is administered at least seven days after the dose of the radiation therapy. In certain embodiments, the dendritic cell activating molecule induces maturation of an immature dendritic cell. In certain embodiments, the dendritic cell activating molecule activates dendritic cell activation through a toll-like receptor, a NOD-like receptor, a RIG-1 or MDA-5 receptor, a C-type lectin receptor, a costimulatory molecule, a cytokine receptor, or a STING pathway. In certain embodiments, the dendritic cell activating molecule is a toll-like receptor agonist selected from the list consisting of CpG oligonucleotide, SD-101, LFX453, imiquimod, Bacillus Calmette-Guérin (BCG), monophosphoryl lipid A, Poly ICLC, GSK1795091, and combinations thereof. In certain embodiments, the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof. In certain embodiments, the dendritic cell activating molecule is a RIG-1 or MDA-5 receptor agonist selected from the list consisting of poly(I:C), Poly(dA:dT), Poly(dG:dC), 3p-hpRNA, 5′ppp-dsRNA, and combinations thereof. In certain embodiments, the dendritic cell activating molecule is a C-type lectin receptor agonist selected from the list consisting of Beta-1,3-glucan, zymosan, Heat-killed C. albicans, cord factor, and Trehalose-6,6-dibehenate, and combinations thereof. In certain embodiments, the dendritic cell activating molecule is a costimulatory molecule agonist selected from the list consisting of a CD40 agonist, aCD80 agonist, a CD86 agonist, an OX40 agonist, and combinations thereof. In certain embodiments, the CD40 agonist is an anti-CD40 agonistic antibody. In certain embodiments, the anti-CD40 agonistic antibody comprises dacetuzumab, CP-870,893, ADC-1013, 2141-v11, APX005M, Chi Lob 7/4, BG9588 (NIAMS), CFZ533, PG10, BMS-986004, lucatumumab, HCD122, JNJ-64457107, selicrelumab, ASKP1240, or SEA-CD40. In certain embodiments, the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof. In certain embodiments, the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB1891, E-7766, or SB11285, and combinations thereof. In certain embodiments, the dendritic cell activating molecule is administered to a tumor being treated with the dose of the radiation therapy. In certain embodiments, the tumor or cancer is a solid tissue tumor or cancer. In certain embodiments, the solid tumor or cancer is of breast, prostate, or a melanoma.

Described herein in one aspect is a method of treating a tumor or a cancer in an individual, the method comprising administering to the individual a dose of an energy-based therapy and a dendritic cell activating molecule, wherein the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy. Also described is a method of treating a tumor or a cancer in an individual, the method comprising administering to the individual a dendritic cell activating molecule, wherein the individual has been administered a dose of an energy-based therapy, wherein the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy. In certain embodiments, the dose of the energy base therapy comprises a plurality of doses of energy-based therapy. In certain embodiments, the energy-based therapy is Irreversible Electroporation (IRE). In certain embodiments, the energy-based therapy is microwave therapy In certain embodiments, the energy-based therapy is Low-Intensity Focused Ultrasound (LOFU). In certain embodiments, the LOFU is administered at an intensity of between 10 and 1000 W/cm² in the area of treatment. In certain embodiments, the energy-based therapy is High-Intensity Focused Ultrasound (HIFU). In certain embodiments, the energy-based therapy is cryotherapy. In certain embodiments, the dendritic cell activating molecule is administered at least three days after the dose of the energy-based therapy. In certain embodiments, the dendritic cell activating molecule is administered at least five days after the dose of the energy-based therapy. In certain embodiments, the dendritic cell activating molecule is administered at least seven days after the dose of the energy-based therapy. In certain embodiments, the dendritic cell activating molecule activates maturation of an immature dendritic cell. In certain embodiments, the dendritic cell activating molecule activates dendritic cell activation through a toll-like receptor, a NOD-like receptor, a RIG-1 or MDA-5 receptor, a C-type lectin receptor, a costimulatory molecule, a cytokine receptor, or a STING pathway. In certain embodiments, the dendritic cell activating molecule is a toll-like receptor agonist selected from the list consisting of CpG oligonucleotide, SD-101, LFX453, imiquimod, Bacillus Calmette-Guérin (BCG), monophosphoryl lipid A, Poly ICLC, GSK1795091, and combinations thereof. In certain embodiments, the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof. In certain embodiments, the dendritic cell activating molecule is a RIG-1 or MDA-5 receptor agonist selected from the list consisting of poly(I:C), Poly(dA:dT), Poly(dG:dC), 3p-hpRNA, 5′ppp-dsRNA, and combinations thereof. In certain embodiments, the dendritic cell activating molecule is a C-type lectin receptor agonist selected from the list consisting of Beta-1,3-glucan, zymosan, Heat-killed C. albicans, cord factor, and Trehalose-6,6-dibehenate, and combinations thereof. In certain embodiments, the dendritic cell activating molecule is a costimulatory molecule agonist selected from the list consisting of a CD40 agonist, aCD80 agonist, a CD86 agonist, an OX40 agonist, and combinations thereof. In certain embodiments, the CD40 agonist is an anti-CD40 agonistic antibody. In certain embodiments, the anti-CD40 agonistic antibody comprises dacetuzumab, CP-870,893, ADC-1013, 2141-v11, APX005M, Chi Lob 7/4, BG9588 (NIAMS), CFZ533, PG10, BMS-986004, lucatumumab, HCD122, JNJ-64457107, selicrelumab, ASKP1240, or SEA-CD40. In certain embodiments, the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof. In certain embodiments, the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB1891, E-7766, or SB11285, and combinations thereof. In certain embodiments, the dendritic cell activating molecule is administered to a tumor being treated with the dose of the energy-based therapy. In certain embodiments, the tumor or cancer is a solid tissue tumor or cancer. In certain embodiments, the solid tumor or cancer is of breast, prostate, or a melanoma.

In one aspect described herein is a method of increasing T cell infiltration into a tumor distal to a tumor being treated in an individual, the method comprising administering to the individual a dose of a radiation therapy and a dendritic cell activating molecule, wherein the dendritic cell activating molecule is administered at least one day after the radiation therapy is administered. In certain embodiments, the dendritic cell activating molecule is administered at least two days after the radiation therapy is administered. In certain embodiments, the dendritic cell activating molecule is administered at least three days after the radiation therapy is administered. In certain embodiments, the dose of the radiation therapy comprises a plurality of doses of radiation therapy. In certain embodiments, the radiation therapy is external beam radiation therapy. In certain embodiments, the external beam radiation therapy is selected from the list consisting of: three-dimensional conformal radiation therapy, intensity modulated radiation therapy, image guided radiation therapy, stereotactic radiation therapy, intraoperative radiation therapy, proton beam therapy, neutron beam therapy, and combinations thereof. In certain embodiments, the dose of radiation therapy comprises at least about 2 Gy. In certain embodiments, the dose of radiation therapy comprises at least about 2 Gy and no more than about 20 Gy. In certain embodiments, the dendritic cell activating molecule is administered at least three days after the dose of the radiation therapy. In certain embodiments, wherein the dendritic cell activating molecule is administered at least five days after the dose of the radiation therapy. In certain embodiments, the dendritic cell activating molecule is administered at least seven days after the dose of the radiation therapy. In certain embodiments, the dendritic cell activating molecule activates maturation of an immature dendritic cell. In certain embodiments, wherein the dendritic cell activating molecule activates dendritic cell activation through a toll-like receptor, a NOD-like receptor, a RIG-1 or MDA-5 receptor, a C-type lectin receptor, a costimulatory molecule, a cytokine receptor, or a STING pathway. In certain embodiments, the dendritic cell activating molecule is a toll-like receptor agonist selected from the list consisting of CpG oligonucleotide, SD-101, LFX453, imiquimod, Bacillus Calmette-Guérin (BCG), monophosphoryl lipid A, Poly ICLC, GSK1795091, and combinations thereof. In certain embodiments, the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof. In certain embodiments, the dendritic cell activating molecule is a RIG-1 or MDA-5 receptor agonist selected from the list consisting of poly(I:C), Poly(dA:dT), Poly(dG:dC), 3p-hpRNA, 5′ppp-dsRNA, and combinations thereof. In certain embodiments, the dendritic cell activating molecule is a C-type lectin receptor agonist selected from the list consisting of Beta-1,3-glucan, zymosan, Heat-killed C. albicans, cord factor, and Trehalose-6,6-dibehenate, and combinations thereof. In certain embodiments, the dendritic cell activating molecule is a costimulatory molecule agonist selected from the list consisting of a CD40 agonist, aCD80 agonist, a CD86 agonist, and combinations thereof. In certain embodiments, the CD40 agonist is an anti-CD40 agonistic antibody. In certain embodiments, the anti-CD40 agonistic antibody comprises dacetuzumab, CP-870,893, ADC-1013, 2141-v11, APX005M, Chi Lob 7/4, BG9588 (NIAMS), CFZ533, PG10, BMS-986004, lucatumumab, HCD122, JNJ-64457107, selicrelumab, ASKP1240, or SEA-CD40. In certain embodiments, the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof. In certain embodiments, the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB1891, E-7766, or SB11285, and combinations thereof. In certain embodiments, the dendritic cell activating molecule is administered to a tumor being treated with the dose of the radiation therapy. In certain embodiments, the tumor is a solid tumor. In certain embodiments, the solid tumor is a breast tumor, a prostate tumor, or a melanoma.

### BRIEF DESCRIPTION OF THE DRAWINGS

FIG. 1A illustrates the experimental protocol for mice that received αCD40 concurrently with radiation treatment.

FIG. 1B illustrates the change in tumor volume over time in control mice and mice that received αCD40 concurrently with radiation treatment.

FIG. 1C illustrates the change in tumor volume over time in individual mice that received radiation only, and mice that received αCD40 concurrently with radiation treatment.

FIG. 2A illustrates the experimental protocol for treatment of tumor bearing mice in tumor-specific T-cell compromised mice that received αCD40 concurrently with radiation treatment.

FIG. 2B illustrates the change in tumor volume over time in tumor-specific T-cell compromised mice that received no treatment, mice that received radiation only, and mice that received αCD40 concurrently with radiation treatment.

FIG. 2C illustrates the change in tumor volume over time in individual tumor-specific T-cell compromised mice that received no treatment, mice that received radiation only, and mice that received αCD40 concurrently with radiation treatment.

FIG. 3A illustrates the experimental protocol for RES499 tumor bearing mice that received αCD40 treatment after radiation treatment.

FIG. 3B illustrates the increase in survival in mice that received αCD40 treatment after radiation treatment compared to both mice that received no treatment or mice that received radiation treatment only.

FIG. 3C illustrates the change in tumor size over time in individual mice that received αCD40 treatment after radiation, mice that received radiation treatment only, and untreated mice.

FIG. 3D illustrates the survival at 100 days post tumor injection of mice that received αCD40 treatment after radiation treatment, mice that received radiation only, and untreated mice.

FIG. 3E illustrates the experimental protocol for the tumor re-challenge experiment.

FIG. 3F illustrates the tumor incidence rate in re-challenged mice that received αCD40 treatment after radiation treatment, mice that received radiation treatment only, and untreated mice.

FIG. 4A illustrates the development of the RES499 cancer cell line.

FIG. 4B illustrates the growth of RES499-derived tumors in mice that received both radiation and αCTLA-4 treatment.

FIG. 4C illustrates that elevated IFNγ signaling in RES499 cells led to increased expression of PDL1 compared to parental cells.

FIG. 4D illustrates the experimental protocol used to assess the growth of the abscopal tumors in a checkpoint blockade (αCTLA4) resistant cell line (RES499).

FIG. 4E illustrates the mean tumor growth in abscopal tumors of mice that received radiation treatment, mice that received either αCTLA-4 or αCD40 treatment in addition to radiation treatment, and untreated mice.

FIG. 4F illustrates the individual tumor growth in both primary and abscopal tumors in control mice, irradiated mice, and mice that received both αCD40 treatment and radiation.

FIGS. 5A-5D illustrate the effect of treatment on co-stimulatory molecule expression and type 1 inflammation in CD103⁺ dendritic cells.

FIGS. 5E-5H illustrate the effect of treatment on co-stimulatory molecule expression and type 1 inflammation in myeloid derived suppressor cells.

FIGS. 5I-5K illustrate the effect of αCD40 treatment on inducible nitric oxide synthetase in myeloid cells, dendritic cells and myeloid derived suppressor cells.

FIGS. 6A-6C illustrate activation-associated co-stimulation in the CD11b⁺ population in the draining lymph node.

FIG. 6D illustrates that IL6 was reduced in mice that received αCD40 after radiation therapy compared to mice that received radiation therapy alone.

FIGS. 6E-6F illustrate the infiltration and levels of MHC class II in granulocytic myeloid derived suppressor cells from mice which received αCD40 after radiation therapy.

FIGS. 6G-6H illustrate the infiltration and levels of MHC class II in monocytic myeloid derived suppressor cells in mice that received αCD40 after radiation therapy.

FIGS. 7A-7B illustrate the effect of αCD40 treatment following radiation therapy on the CD4/CD8 ratio.

FIG. 7C illustrates the effect of αCD40 treatment following radiation therapy on regulatory T cells.

FIGS. 7D-7E illustrate the effect of αCD40 treatment following radiation therapy on IFNγ⁺ CD8 cells.

FIGS. 7F-7G illustrate the effect of αCD40 treatment following radiation therapy on effector CD8 T cell proliferation.

FIG. 8A illustrates the effect of αCD40 treatment following radiation therapy on the CD4/CD8 ratio in draining lymph nodes.

FIG. 8B illustrates the effect of αCD40 treatment following radiation therapy on Ki67⁺ cells.

FIG. 8C illustrates the effect of αCD40 treatment following radiation therapy on the percent of CD44⁺ CD8 cells.

FIG. 8D illustrates the effect of αCD40 treatment following radiation therapy on T cells.

FIG. 8E illustrates the effect of αCD40 treatment following radiation therapy on natural killer cells.

FIG. 8F illustrates the effect of αCD40 treatment following radiation therapy on Foxp3⁺ CD4 cells.

FIG. 8G illustrates the effect of αCD40 treatment following radiation therapy on the percent of IFN⁺ CD8 cells.

FIG. 8H illustrates the effect of αCD40 treatment following radiation therapy on central memory.

FIG. 9A illustrates the experimental protocol used to test the effect of αCD40 treatment following radiation therapy in a metastatic cancer model.

FIG. 9B illustrates survival of mice treated with αCD40 following radiation in a metastatic cancer model.

FIG. 9C illustrates a comparison of the survival rates of different groupings of treatment types of mice treated with αCD40 following radiation in a metastatic cancer model as in FIG. 9C.

FIG. 10A illustrates the experimental protocol used to treat survival of mice treated with αCD40 following radiation in a melanoma cancer model.

FIG. 10B illustrates the tumor volume of individual mice inoculated with B16F10 cells (top panels) or RES499 cells (bottom panels) treated with αCD40 following radiation.

FIG. 11 illustrates the course of treatment of a patient with cancer with PAM and additional therapies.

FIG. 12A depicts the experimental protocol used to test the effects or anti-CD40 therapy and irradiation on exhaustion of tumor-infiltrating T-cells.

FIG. 12B depicts flow cytometry analysis of cell types in mice after treatment.

FIG. 12C depicts a comparison of the percent of GrBz+Ki67+ cells in each treatment group.

FIG. 13A depicts the experimental protocol used to test the effects of depletion of immune cells.

FIG. 13B depicts the effect of depletion of CD8 cells on tumor volume.

FIG. 13C depicts the effects of depletion of Ly6C and CD11b cells on tumor volume.

### DETAILED DESCRIPTION

Disclosed herein is a method of treating a tumor or a cancer in an individual by administering a dendritic cell activating molecule to an individual at least one day after treatment with either radiation therapy or an energy therapy. Both radiation therapy and energy therapies treat tumors and cancers in individuals by killing or damaging the cancer cells. The addition of administering a dendritic cell activating molecule activates the dendritic cells of the individual’s immune system and aids in treating the tumor or cancer.

In one aspect described herein is a method comprising administering to the individual a dose of a radiation therapy and a dendritic cell activating molecule, wherein the dendritic cell activating molecule is administered at least one day after the radiation therapy is administered. In another aspect described herein is a method comprising administering to the individual a dendritic cell activating molecule, wherein the individual has received a dose of a radiation therapy, and wherein the dendritic cell activating molecule is administered at least one day after the radiation therapy has been administered.

In one aspect described herein is a method comprises administering to the individual a dose of an energy-based therapy and a dendritic cell activating molecule, wherein the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy. In another aspect described herein is a method comprising administering to the individual a dendritic cell activating molecule, wherein the individual has been administered a dose of an energy-based therapy, wherein the dose of the energy based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy.

This application also discloses a method of increasing T cell infiltration into a tumor distal to a tumor being treated in an individual. In one aspect described herein is a method comprising administering to the individual a dose of a radiation therapy and a dendritic cell activating molecule, wherein the dendritic cell activating molecule is administered at least one day after the radiation therapy is administered. In another aspect described herein is a method comprising administering to the individual a dose of an energy-based therapy and a dendritic cell activating molecule, wherein the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy.

### Certain Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. Compositions for treating or preventing a given disease can consist essentially of the recited active ingredient, exclude additional active ingredients, but include other non-material components such as excipients, carriers, or diluents. “Consisting of” shall mean excluding more than trace elements of other ingredients and substantial method steps. Embodiments defined by each of these transition terms are within the scope of this disclosure.

As used herein the term “about” refers to an amount that is near the stated amount by 10%.

As used herein the terms “individual,” “patient,” or “subject” are used interchangeably and refer to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

As used herein the term “treat” or “treating” refers to interventions to a physiological or disease state of an individual designed or intended to ameliorate at least one sign or symptom associated with said physiological or disease state. The skilled artisan will recognize that given a heterogeneous population of individuals afflicted with a disease, not all individuals will respond equally, or at all, to a given treatment.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “radiotherapy” or “radiation therapy” means the treatment of an individual with ionizing radiation. Exemplary types of radiation therapy include without limitation three-dimensional conformal radiation therapy, intensity modulated radiation therapy, image guided radiation therapy, stereotactic radiation therapy, intraoperative radiation therapy, proton beam therapy, and neutron beam therapy.

The term “energy-based therapy” means the treatment of an individual with a form of energy, including without limitations electrical currents, electromagnetic waves, and temperature. Exemplary types of energy-based therapy include without limitation Irreversible Electroporation (IRE), Microwave, Low-Intensity Focused Ultrasound (LOFU), High-Intensity Focused Ultrasound (HIFU), Radiofrequency energy, and cryotherapy.

The term “immune cell” refers to a cell that plays a role in the immune response and originates from a hematopoietic precursor. Without limitation, immune cells include lymphocytes, such as B cells and T cells; natural killer cells; and myeloid cells, such as monocytes, macrophages, eosinophils, mast cells, basophils, dendritic cells, and granulocytes.

The term “dendritic cell” refers to an antigen-presenting cell of the immune system of hematopoietic origin. Dendritic cells can be characterized by the expression of class II MHC, CD11c and CD86. Dendritic cells include without limitation activated dendritic cells, non-activated dendritic cells, mature dendritic cells, and immature dendritic cells.

The term “dendritic cell activating molecule” refers to a molecule that increases the immunological activity of dendritic cells as compared to the dendritic cell activity prior to exposure to the activating agent. Changes in the immunological activity of dendritic cells may include without limitation changes to antigen presentation, migration to lymph nodes, interaction with T cells and B cells, T-cell priming, cytokine release, and chemokine release. Examples of dendritic cell activating molecules include, without limitation, CD40L, an anti-CD40 agonist antibody, a TLR activator, a NOD-like receptor agonist, a RIG-1 receptor agonist, an MDA-5 receptor agonist, a C-type lectin receptor agonist, a STING activator, a costimulatory molecule or a cytokine receptor. Other suitable activating molecules useful in the practice of the methods described herein include a RANKL peptide, TNF peptide, IL-1 peptide, CpG-rich DNA sequences, lipopolysaccharide (LPS), RIG1 helicase ligand, RNA, dsDNA or variations thereof (e.g., polypeptides or DNA sequences comprising one or more insertions, substitutions, or deletions).

The term “antibody” as used herein refers to polypeptides comprising at least one antibody derived antigen binding site (e.g., VH/VL region or Fv, or CDR), and includes whole antibodies and any antigen binding fragments (i.e., “antigen-binding portions” or antigen binding fragments thereof) or single chains thereof. Antibodies include known forms of antibodies. For example, the antibody can be a human antibody, a humanized antibody, a bispecific antibody, or a chimeric antibody. A “whole antibody” refers to a glycoprotein comprising at least two heavy (H) chains and two light (L) chains inter-connected by disulfide bonds, in which each heavy chain is comprised of a heavy chain variable region (abbreviated herein as VH) and a heavy chain constant region; and each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. An “antigen-binding fragment” includes without limitations Fab, Fab′, F(ab′)₂, scFv, Fv, recombinant IgG, and heavy chain antibodies.

The term “tumor,” or “cancer” as used herein, and unless otherwise specified, refers to a neoplastic cell growth, and includes pre-cancerous and cancerous cells and tissues. Tumors usually present as a lesion or lump. As used herein, “treating” a tumor means that one or more symptoms of the disease, such as the tumor itself, vascularization of the tumor, or other parameters by which the disease is characterized, are reduced, ameliorated, inhibited, placed in a state of remission, or maintained in a state of remission. “Treating” a tumor also means that one or more hallmarks of the tumor may be eliminated, reduced or prevented by the treatment. Non-limiting examples of such hallmarks include uncontrolled degradation of the basement membrane and proximal extracellular matrix, migration, division, and organization of the endothelial cells into new functioning capillaries, and the persistence of such functioning capillaries.

### Radiation Therapies

### Types of Radiation Therapies

The methods described herein comprise or consist essentially of administering a radiation therapy and a dendritic cell activator to an individual in need thereof. Any of the radiation therapies described herein can be administered either alone or in combination. Radiation therapies described herein can be administered either singly or as plurality of doses.

In general, radiation therapy, radio-immunotherapy or pre-targeted radioimmunotherapy are used for the treatment of diseases of oncological nature. “Radiotherapy” or radiation therapy means the treatment of cancer and other diseases with ionizing radiation. Ionizing radiation deposits energy that injures or destroys cells in the area being treated (the target tissue) by damaging their genetic material, making it impossible for these cells to continue to grow. Radiotherapy may be used to treat localized solid tumors, such as cancers of the skin, tongue, larynx, brain, breast, lung, liver, kidney, pancreas, or uterine cervix. It can also be used to treat leukemia and lymphoma, i.e. cancers of the blood-forming cells and lymphatic system, respectively. In certain aspects of the methods disclosed herein, radiation therapy is used to treat a tumor.

Ionizing radiation is widely used for the treatment of solid tumors. Several types of ionizing radiation can be used, including X-rays and gamma rays. Radiotherapy can be applied using a machine to focus the radiation on the tumor, or by placing radioactive implants directly into the tumor or in a nearby body cavity. Moreover, radiolabeled antibodies can be used to target tumor cells. Other radiotherapy techniques may also be used in the methods described herein, including intraoperative irradiation, particle beam radiation, as well as the use of radiosensitizers to make tumor cells more sensitive to radiation, or radioprotectants to protect normal cells.

One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons are machines that produce x-rays of increasingly greater energy. The use of machines to focus radiation (such as x-rays) on a cancer site is called external beam radiotherapy. In one embodiment of the methods, external beam radiotherapy is used.

Three-dimensional conformal radiation therapy, intensity-modulated radiation therapy, and image-guided radiation therapy are methods of external beam radiotherapy that allow for more precise targeting of the tumor while avoiding more of the surrounding healthy issue. The increased precision allows for higher levels of radiation, which is more effective in shrinking and killing tumors. In three -dimensional conformal radiation therapy, targeting information is used to shape the radiation beam to the shape of the tumor. In image-guided radiation therapy, computer-controlled linear accelerators are used to target specific areas within a tumor. This method allows the radiation dose to more closely match the shape of the tumor by controlling the intensity of the beam in multiple small volumes. Image-guided radiation therapy uses imaging during the radiation therapy to improve the precision and accuracy of treatment. Imaging methods include but are not limited to fiducial markers, ultrasound, MRI, x-ray images, CT-scan, 3-D body surface mapping, electromagnetic transponders, or colored tattoos. Image-guided radiation therapy is especially useful in tumors located in areas of the body that move, such as the lungs. In one embodiment of the methods, three-dimensional conformal radiation therapy is used. In another embodiment of the methods, intensity-modulated radiation therapy is used. In another embodiment of the methods, image-guided radiation therapy is used.

High dose radiotherapy, such as stereotactic ablative radiotherapy (SABR) or stereotactic body radiation therapy (SBRT), is another method of external beam radiation radiotherapy. Higher doses, in the range of 15 to 20 Gy are used than in convention radiotherapy. One type of SABR is stereotactic radiosurgery (SRS), which has been used for small intracranial tumors that was made possible by technology allowing for submillimeter delivery precision and steep dose gradients beyond the tumor target. SABR (or SBRT) has been developed for use on tumors outside of the brain and includes tumors of practically every major body site (e.g., lung tumors). In one embodiment, the external beam radiation therapy is stereotactic ablative radiotherapy.

Another method of external beam radiotherapy is intraoperative irradiation, in which a large dose of external radiation is directed at the tumor and surrounding tissue during surgery. In one embodiment, the external beam radiation is intraoperative irradiation.

Gamma rays are another form of photons used in radiotherapy. Gamma rays are produced spontaneously as certain elements (such as radium, uranium, and cobalt 60) release radiation as they decompose or decay. In one embodiment, the external beam radiation is gamma ray radiation.

Another approach is particle beam radiation therapy. This type of therapy differs from photon radiotherapy in that it involves the use of fast-moving subatomic particles to treat localized cancers. This includes, but is not limited to, proton beam therapy, neutron beam therapy, pion beam therapy, and heavy ion beam therapy. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation. Radio-sensitizers make the tumor cells more likely to be damaged, and radio-protectors protect normal tissues from the effects of radiation. In one embodiment, the external beam radiation is selected from the list consisting of proton beam therapy, neutron beam therapy, pion beam therapy, and heavy ion beam therapy. In one embodiment the external beam radiation used is proton beam therapy. In another embodiment, the external beam therapy used is neutron beam therapy. In another embodiment, the external beam therapy used is pion beam therapy. In another embodiment, the external beam therapy used is heavy ion beam therapy. In one embodiment, the external beam radiation therapy is selected from the list consisting of: three-dimensional conformal radiation therapy, intensity modulated radiation therapy, image guided radiation therapy, stereotactic radiation therapy, intraoperative radiation therapy, proton beam therapy, neutron beam therapy, and combinations thereof.

Another technique for delivering radiation to cancer cells is to place radioactive implants directly in a tumor or body cavity. This is called internal radiotherapy. Brachytherapy, interstitial irradiation, and intracavitary irradiation are types of internal radiotherapy. In this treatment, the radiation dose is concentrated in a small area, and the patient stays in the hospital for a few days. Internal radiotherapy is frequently used for cancers of the tongue, uterus, and cervix. In one embodiment, internal radiotherapy is used. In another embodiment, the internal radiotherapy is selected from the list comprising brachytherapy, interstitial irradiation, and intracavitary irradiation, or combinations thereof.

### Doses of Radiation Therapies

In certain cases, the total irradiation dose can be spread over several sessions (i.e., dose fractionation) and can be spaced by at least 6 hours, days, or even weeks. Conventional definitive radiation treatment involves multiple treatments, generally 20-40, with low doses (<2- 3 Gy) stretching over weeks. In certain cases, such as high doses radiotherapy discussed above, the dose is greater than 15-20 Gy and is given is up to 5 treatments.

Certain aspects of the method disclosed herein comprise treating a patient with radiotherapy. In one embodiment, the method includes a plurality of doses of radiation therapy. In one embodiment, the method includes at least 2 doses of radiation therapy. In another embodiment, the method includes at least 3 doses of radiation therapy. In another embodiment, the method includes at least 4 doses of radiation therapy. In another embodiment, the method includes at least 5 doses of radiation therapy. In another embodiment the method includes at least 6 doses of radiation therapy. In another embodiment, the method includes at least 7 doses of radiation therapy. In another embodiment, the method includes at least 8 doses of radiation therapy. In another embodiment, the method includes at least 9 doses of radiation therapy. In another embodiment, the method includes at least 10 doses of radiation therapy. In another embodiment, the method includes at least 11 doses of radiation therapy. In another embodiment, the method includes at least 12 doses of radiation therapy. In another embodiment, the method includes at least 13 doses of radiation therapy. In another embodiment, the method includes at least 14 doses of radiation therapy. In another embodiment, the method includes at least 15 doses of radiation therapy. In another embodiment, the method includes at least 20 doses of radiation therapy. In another embodiment, the method includes at least 25 doses of radiation therapy. In another embodiment, the method includes at least 30 doses of radiation therapy. In another embodiment, the method includes at least 35 doses of radiation therapy. In another embodiment, the method includes at least 40 doses of radiation therapy. In another embodiment, the method includes at least 45 doses of radiation therapy. In another embodiment, the method includes at least 50 doses of radiation therapy.

In one aspect of the methods described herein, the radiation therapy uses ionizing radiation for treating cancer in a subject. In one embodiment, the dose of radiation therapy is at least about 2 Gy. In another embodiment, the dose of radiation therapy is at least about 3 Gy. In another embodiment, the dose of radiation therapy is at least about 4 Gy. In another embodiment, the dose of radiation therapy is at least about 5 Gy. In another embodiment, the dose of radiation therapy is at least about 6 Gy. In another embodiment, the dose of radiation therapy is at least about 7 Gy. In another embodiment, the dose of radiation therapy is at least about 8 Gy. In another embodiment, the dose of radiation therapy is at least about 9 Gy. In another embodiment, the dose of radiation therapy is at least about 10 Gy. In another embodiment, the dose of radiation therapy is at least about 15 Gy. In another embodiment, the dose of radiation therapy is at least about 20 Gy. In another embodiment, the dose of radiation therapy is at least about 25 Gy. In another embodiment, the dose of radiation therapy is at least about 30 Gy. In another embodiment, the dose of radiation therapy is at least about 40 Gy. In another embodiment, the dose of radiation therapy is at least about 50 Gy. In another embodiment, the dose of radiation therapy is at least about 60 Gy. In another embodiment, the dose of radiation therapy is at least about 70 Gy. In another embodiment, the dose of radiation therapy is at least about 80 Gy. In another embodiment, the dose of radiation therapy is at least about 90 Gy. In another embodiment, the dose of radiation therapy is at least about 100 Gy.

In one embodiment, the total radiation dose for a cycle of treatment is between 5 and 100 Gy. In another embodiment, the total radiation dose for a cycle of treatment is between about 10 and about 100 Gy. In another embodiment, the total radiation dose for a cycle of treatment is between about 20 and about 100 Gy. In another embodiment, the total radiation dose for a cycle of treatment is between about 30 and about 100 Gy. In another embodiment, the total radiation dose for a cycle of treatment is between about 40 and about 100 Gy. In another embodiment, the total radiation dose for a cycle of treatment is between about 50 and about 100 Gy. In another embodiment, the total radiation dose for a cycle of treatment is between about 60 and about 100 Gy. In another embodiment, the total radiation dose for a cycle of treatment is between about 70 and about 100 Gy. In another embodiment, the total radiation dose for a cycle of treatment is between about 80 and about 100 Gy. In another embodiment, the total radiation dose for a cycle of treatment is between about 90 and about 100 Gy. In another embodiment, the total radiation dose for a cycle of treatment is about 100 Gy.

In one embodiment, the total radiation dose for a cycle of treatment is between about 20 to about 50 Gy. In one embodiment, the total radiation dose for a cycle of treatment is between about 20 to about 50 Gy on one occasion. In another embodiment, the total radiation dose for a cycle of treatment is between about 20 to about 50 Gy on each of two occasions. In another embodiment, the total radiation dose for a cycle of treatment is between about 10 to about 30 Gy. In another embodiment, the total radiation dose for a cycle of treatment is between about 10 to about 30 Gy on one occasion. In another embodiment, the total radiation dose for a cycle of treatment is between about 10 to about 30 Gy on each of two occasions. In another embodiment, the total radiation dose for a cycle of treatment is between about 10 to about 30 Gy on each of three occasions. In another embodiment, the total radiation dose for a cycle of treatment is between about 10 to about 30 Gy on each of four occasions. In another embodiment, the total radiation dose for a cycle of treatment is between about 10 to about 30 Gy on each of five occasions. In another embodiment, the total radiation dose for a cycle of treatment is between about 10 to about 30 Gy on each of two to four occasions.

In another embodiment, the total radiation dose for a cycle of treatment is between about 5 and about 20 Gy. In another embodiment, the total radiation dose for a cycle of treatment is between about 5 and about 20 Gy on one occasion. In another embodiment, the total radiation dose for a cycle of treatment is between about 5 and about 20 Gy on each of two occasions. In another embodiment, the total radiation dose for a cycle of treatment is between about 5 and about 20 Gy on each of three occasions. In another embodiment, the total radiation dose for a cycle of treatment is between about 5 and about 20 Gy on each of four occasions. In another embodiment, the total radiation dose for a cycle of treatment is between about 5 and about 20 Gy on each of five occasions. In a certain embodiments, the total radiation dose for a cycle of treatment is between about 20 and about 50 Gy on one occasion, between about 10 and about 30 Gy on each of two to four occasions, or between about 5 and about 20 Gy on each of 5 occasions. In another embodiment, the total radiation dose for a cycle of treatment is between about 30 to about 40 Gy on one occasion. In another embodiment, the total radiation dose for a cycle of treatment is between about 30 to about 40 Gy on each of two occasions. In another embodiment, the total radiation dose for a cycle of treatment is between about 15 to about 20 Gy on one occasion. In another embodiment, the total radiation dose for a cycle of treatment is between about 15 to about 20 Gy on each of two occasions. In another embodiment, the total radiation dose for a cycle of treatment is between about 15 to about 20 Gy on each of three occasions. In another embodiment, the total radiation dose for a cycle of treatment is between about 15 to about 20 Gy on each of four occasions. In another embodiment, the total radiation dose for a cycle of treatment is between about 8 to about 12 Gy on one occasion. In another embodiment, the total radiation dose for a cycle of treatment is between about 8 to about 12 Gy on each of two occasions. In another embodiment, the total radiation dose for a cycle of treatment is between about 8 to about 12 Gy on each of three occasions. In another embodiment, the total radiation dose for a cycle of treatment is between about 8 to about 12 Gy on each of four occasions. In another embodiment, the total radiation dose for a cycle of treatment is between about 8 to about 12 Gy on each of five occasions. In another embodiment, the total radiation dose for a cycle of treatment is between about 8 to about 12 Gy on each of six occasions. In a certain embodiments, the total radiation dose for a cycle of treatment is between about 30 to about 40 Gy on one occasion, about 15 to about 20 Gy on each of three occasions, or about 8 to about 12 Gy on each of 5 occasions.

The methods described herein may also be combined with post-ablation modulation (PAM) after high dose radiation. PAM can be administered from about 0.1 Gy to about 2 Gy, from about 0.1 Gy to about 1 Gy, from about 0.2 to about to about 2 Gy, from about 0.1 to about to about 0.8 Gy, from about 0.1 to about to about 0.6 Gy, from about 0.2 to about to about 0.6 Gy, from about 0.4 to about to about 0.6 Gy. PAM can be administered at about 0.1, 0.2, 0.4, 0.5, 0.6, 0.8, or 1.0 Gy. PAM can be administered for about 1, 2, 3, 4, 5, 6, 7, 8, 9, 10 or more doses.

### Energy-Based Therapy

The methods described herein comprise or consist essentially of administering an energy-based therapy and a dendritic cell activator to an individual in need thereof. Any of the energy based therapies described herein can be administered either alone or in combination. Energy-based therapies described herein can be administered either singly or a plurality of times.

A variety of energy-based therapies can be administered to treat cancer. These methods use electromagnetic waves, electromagnetic currents or temperature to kill or damage cancer or tumor cells. These include, but are not limited to, Irreversible Electroporation (IRE), Microwave, Low-Intensity Focused Ultrasound (LOFU), High-Intensity Focused Ultrasound (HIFU), Radiofrequency energy, and cryotherapy. In one aspect of the methods disclosed herein, the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy.

Certain aspects of the methods disclosed herein involve treating a patient with an energy-based therapy. In one embodiment, the dose of the energy-based therapy comprises a plurality of doses of energy-based therapy. In one embodiment, the dose of the energy-based therapy may comprise at least 2 doses. In another embodiment, the dose of the energy-based therapy may comprise at least 3 doses. In another embodiment, the dose of the energy-based therapy may comprise at least 4 doses. In another embodiment, the dose of the energy-based therapy may comprise at least 5 doses. In another embodiment, the dose of the energy-based therapy may comprise at least 6 doses. In another embodiment, the dose of the energy-based therapy may comprise at least 7 doses. In another embodiment, the dose of the energy-based therapy may comprise at least 8 doses. In another embodiment, the dose of the energy-based therapy may comprise at least 9 doses. In another embodiment, the dose of the energy-based therapy may comprise at least 10 doses. In another embodiment, the dose of the energy-based therapy may comprise more than 10 doses.

Irreversible Electroporation (IRE) is a method of treating a tumor that uses electrical currents to damage and destroy cancer cells. Electrodes are placed around the tumor and a current is delivered through the electrodes. The application of the current results in permeabilization of the cell membrane, resulting in apoptosis of the cancer cells. In one embodiment, the energy-based therapy is Irreversible Electroporation (IRE).

Treatment of localized tumors by focused ultrasound (FUS) is an image guided minimally invasive therapy that uses a range of input energy for in situ tumor ablation. The application of FUS to biological tissues is associated with the generation of thermal and cavitation effects, causing changes in target cell physiology, depending on the energy delivered. High intensity focused ultrasound (HIFU) has been used clinically to thermally ablate localized tumors. The substantial thermal energy generated by that modality of FUS treatment causes rapid coagulative necrosis of the tissue at the targeted focal spots. Though several studies have reported some immunomodulatory effects, including increased lymphocyte infiltration, generation of IFNγ producing tumor-specific T cells in lymphoid organs and dendritic cell maturation and migration into tumors, the thermally induced coagulative necrosis resulting from HIFU treatment can also attenuate the release of immunostimulatory molecules within the tumor microenvironment. Thus, although able to halt the progression of established primary tumors, HIFU might fail to protect against local and distant metastases arising from the surviving tumor cells. In one embodiment, the energy-based therapy is High-Intensity Focused Ultrasound (HIFU).

In some embodiments, HIFU is administered with an intensity of about 100 to about 10000 W/cm² in the area of treatment. In some embodiments, HIFU is administered with an intensity of about 1000 to about 2000 W/cm² in the area of treatment. In some embodiments, HIFU is administered with an intensity of about 2000 to about 3000 W/cm² in the area of treatment. In some embodiments, HIFU is administered with an intensity of about 3000 to about 4000 W/cm² in the area of treatment. In some embodiments, HIFU is administered with an intensity of about 4000 to about 5000 W/cm² in the area of treatment. In some embodiments, HIFU is administered with an intensity of about 5000 to about 6000 W/cm² in the area of treatment. In some embodiments, HIFU is administered with an intensity of about 6000 to about 7000 W/cm² in the area of treatment. In some embodiments, HIFU is administered with an intensity of about 7000 to about 8000 W/cm² in the area of treatment. In some embodiments, HIFU is administered with an intensity of about 8000 to about 9000 W/cm² in the area of treatment. In some embodiments, HIFU is administered with an intensity of about 9000 to about 10000 W/cm² in the area of treatment.

Low energy non-ablative focused ultrasound, or LOFU is an ultrasound treatment, generated using a concave transducer to focus the ultrasound in a treatment zone. Methods and systems for treatment of cancer with LOFU are described in US 202003/98084 and U.S. 10,974,077, which are herein incorporated by reference. LOFU produces mild mechanical and thermal stress in tumor cells, while avoiding cavitation and coagulative necrosis both of which result in tissue damage. A non-ablative “sonic” stress response is induced in the tumor that increases the expression of heat shock proteins without actually killing them directly. In one embodiment, the energy-based therapy is Low-Intensity Focused Ultrasound (LOFU).

In some embodiments, LOFU involves the application of ultrasound at an acoustic power between 10 and 1000 W/cm2 spatial peak temporal average intensity (Ispta) in a treatment zone, with the ultrasound applied continuously for a time in the range of 0.5 to 5 seconds, wherein the frequency is in the range of 0.01 to 10 MHz and the mechanical index is less than 4. Mechanical Index (MI) is the rarefaction pressure in units of MPa over the square root of the central frequency in units of MHz. The energy and intensity of ultrasound applied is intended to fall between energies and intensities of ultrasound that either induce primarily ablative effects or primarily diagnostic effects.

In some embodiments, the LOFU includes a transducer that generates acoustic power between 10 and 1000 W/cm² spatial peak temporal average intensity (Iₛₚta) in a treatment zone. The ultrasound is applied continuously for a time in the range of 0.5 to 5 seconds or pulsed with pulse durations of 1 to 100 ms, wherein the frequency is in the range of 0.01 to 10 MHz. In some embodiments the frequency is in the range of 0.05 to 5 MHz. In some embodiments the frequency range is from 0.1 to 2 MHz. In some embodiments the minimum diameter of any ultrasound beam in the treatment zone is about 1 cm. In an embodiment, the LOFU is administered at 10 to 1000 W/cm² in the area of treatment. In an embodiment, the LOFU is administered at 10 to 100 W/cm² Iₛₚta in the area of treatment. In an embodiment, the LOFU is administered at 100 to 200 W/cm² Iₛₚta in the area of treatment. In an embodiment, the LOFU is administered at 300 to 400 W/cm² Iₛₚta in the area of treatment. In an embodiment, the LOFU is administered at 400 to 500 W/cm² Iₛₚta in the area of treatment. In an embodiment, the LOFU is administered at 500 to 600 W/cm² Iₛₚta in the area of treatment. In an embodiment, the LOFU is administered at 600 to 700 W/cm² Iₛₚta in the area of treatment. In an embodiment, the LOFU is administered at 700 to 800 W/cm² Iₛₚta in the area of treatment. In an embodiment, the LOFU is administered at 800 to 900 W/cm² Iₛₚta in the area of treatment. In an embodiment, the LOFU is administered at 900 to 1000 W/cm² Iₛₚta in the area of treatment. In an embodiment, the ultrasound is applied for a time in the range of 0.5 to 1 second. In an embodiment, the ultrasound is applied for a time in the range of 1 to 2 seconds. In an embodiment, the ultrasound is applied for a time in the range of 2 to 3 seconds. In an embodiment, the ultrasound is applied for a time in the range of 4 to 5 seconds. In embodiment, the ultrasound is applied at a frequency of 0.01 to 1 MHz. In embodiment, the ultrasound is applied at a frequency of 1 to 2 MHz. In embodiment, the ultrasound is applied at a frequency of 2 to 3 MHz. In embodiment, the ultrasound is applied at a frequency of 3 to 4 MHz. In embodiment, the ultrasound is applied at a frequency of 4 to 5 MHz. In embodiment, the ultrasound is applied at a frequency of 5 to 6 MHz. In embodiment, the ultrasound is applied at a frequency of 6 to 7 MHz. In embodiment, the ultrasound is applied at a frequency of 7 to 8 MHz. In embodiment, the ultrasound is applied at a frequency of 8 to 9 MHz. In embodiment, the ultrasound is applied at a frequency of 9 to 10 MHz.

Both microwave therapy and radiofrequency therapy are methods that create localized heat regions to destroy tumors. In radiofrequency therapy, high frequency electrical currents are passed through an electrode placed in a tumor. This creates a small region of heat. In microwave therapy, a needle placed in the tumor creates microwaves which then create a small region of heat. In both treatment methods, the cancer cells within the localized heat region are damaged or destroyed. In one embodiment, the energy-based therapy is microwave therapy. In another embodiment, the energy-based therapy is radiofrequency therapy.

In contrast, cryotherapy is an energy-based therapy uses extreme cold to destroy cancer tissue. Intense cold is created, usually by applying either liquid nitrogen or pressurized argon gas to a localized site. Cells and tissues that encounter the cold are killed. This method can be used on both internal and external tumors. In one embodiment, the energy-based therapy is cryotherapy.

### Dendritic Cell Activating Molecules

The methods described herein comprise or consist essentially of administering: (a) a radiation therapy, an energy based therapy, or a combination thereof; and (b) a dendritic cell activator to an individual in need thereof. Any of the radiation therapies or energy-based therapies described herein can be administered either alone or in combination. Radiation or energy-based therapies described herein can be administered either singly or a plurality of times.

### Timing of Administration

Administration of the dendritic cell activating therapy may be administered at such time as the T cells associated with a with a radiation or energy treated tumor have recovered from the effects of the treatment. Without being bound by theory administration of radiation or energy based therapies disproportionately harms rapidly dividing cells, such as immune cells, and an interval between the administration of a radiation or energy based therapy and a dendritic cell activator may be beneficial to subsequent immune response.

With regard to the timing of a subsequent administration the radiation or energy-based therapy is considered administered on day 0, with the next day after the treatment comprising 1 day after the therapy. Additionally, the amount of days after administration is calculated from the temporally most recent doe of the therapy. Therefore, for example, if an individual is administered a plurality of doses of radiation or energy-based therapy the interval for administration of a dendritic cell activating therapy is calculated based upon the last dose of the plurality before the dendritic cell activating therapy is administered.

In one aspect of the methods disclosed herein, the methods comprise administering a dendritic cell activating molecule after radiation therapy. In one embodiment, the dendritic cell activating molecule is administered at least 1 day after radiation therapy. In another embodiment, the dendritic cell activating molecule is administered at least 2 days after radiation therapy. In another embodiment, the dendritic cell activating molecule is administered at least 3 days after radiation therapy. In another embodiment, the dendritic cell activating molecule is administered at least 4 days after radiation therapy. In another embodiment, the dendritic cell activating molecule is administered at least 5 days after radiation therapy. In another embodiment, the dendritic cell activating molecule is administered at least 6 days after radiation therapy. In another embodiment, the dendritic cell activating molecule is administered at least 7 days after radiation therapy. In another embodiment, the dendritic cell activating molecule is administered at least 8 days after radiation therapy. In another embodiment, the dendritic cell activating molecule is administered at least 9 days after radiation therapy. In another embodiment, the dendritic cell activating molecule is administered at least 10 days after radiation therapy. In another embodiment, the dendritic cell activating molecule is administered at least 11 days after radiation therapy. In another embodiment, the dendritic cell activating molecule is administered at least 12 days after radiation therapy. In another embodiment, the dendritic cell activating molecule is administered at least 13 days after radiation therapy. In another embodiment, the dendritic cell activating molecule is administered at least 14 days after radiation therapy. In another embodiment, the dendritic cell activating molecule is administered more than 14 days after radiation therapy.

In one embodiment, the dendritic cell activating molecule is administered between 1 and 14 days after radiation treatment. In another embodiment, the dendritic cell activating molecule is administered between 2 and 14 days after radiation treatment. In another embodiment, the dendritic cell activating molecule is administered between 3 and 14 days after radiation treatment. In another embodiment, the dendritic cell activating molecule is administered between 4 and 14 days after radiation treatment. In another embodiment, the dendritic cell activating molecule is administered between 5 and 14 days after radiation treatment. In another embodiment, the dendritic cell activating molecule is administered between 6 and 14 days after radiation treatment. In another embodiment, the dendritic cell activating molecule is administered between 7 and 14 days after radiation treatment. In another embodiment, the dendritic cell activating molecule is administered between 8 and 14 days after radiation treatment. In another embodiment, the dendritic cell activating molecule is administered between 9 and 14 days after radiation treatment. In another embodiment, the dendritic cell activating molecule is administered between 10 and 14 days after radiation treatment. In another embodiment, the dendritic cell activating molecule is administered between 11 and 14 days after radiation treatment. In another embodiment, the dendritic cell activating molecule is administered between 12 and 14 days after radiation treatment. In another embodiment, the dendritic cell activating molecule is administered between 13 and 14 days after radiation treatment. In another embodiment, the dendritic cell activating molecule is administered between 1 and 10 days after radiation treatment. In another embodiment, the dendritic cell activating molecule is administered between 2 and 10 days after radiation treatment. In another embodiment, the dendritic cell activating molecule is administered between 3 and 10 days after radiation treatment. In another embodiment, the dendritic cell activating molecule is administered between 4 and 10 days after radiation treatment. In another embodiment, the dendritic cell activating molecule is administered between 5 and 10 days after radiation treatment. In another embodiment, the dendritic cell activating molecule is administered between 6 and 10 days after radiation treatment. In another embodiment, the dendritic cell activating molecule is administered between 7 and 10 days after radiation treatment. In another embodiment, the dendritic cell activating molecule is administered between 8 and 10 days after radiation treatment. In another embodiment, the dendritic cell activating molecule is administered between 9 and 10 days after radiation treatment. In another embodiment, the dendritic cell activating molecule is administered between 1 and 7 days after radiation treatment. In another embodiment, the dendritic cell activating molecule is administered between 2 and 7 days after radiation treatment. In another embodiment, the dendritic cell activating molecule is administered between 3 and 7 days after radiation treatment. In another embodiment, the dendritic cell activating molecule is administered between 4 and 7 days after radiation treatment. In another embodiment, the dendritic cell activating molecule is administered between 5 and 7 days after radiation treatment. In another embodiment, the dendritic cell activating molecule is administered between 6 and 7 days after radiation treatment. In another embodiment, the dendritic cell activating molecule is administered between 1 and 5 days after radiation treatment. In another embodiment, the dendritic cell activating molecule is administered between 2 and 5 days after radiation treatment. In another embodiment, the dendritic cell activating molecule is administered between 3 and 5 days after radiation treatment. In another embodiment, the dendritic cell activating molecule is administered between 4 and 5 days after radiation treatment.

In one aspect of the methods disclosed herein, the methods comprise administering the dendritic cell activating molecules after a dose of an energy-based therapy. In one embodiment, the dendritic cell activating molecule is administered at least 1 day after a dose of the energy-based therapy. In another embodiment, the dendritic cell activating molecule is administered at least 2 days after a dose of the energy-based therapy. In another embodiment, the dendritic cell activating molecule is administered at least 3 days after a dose of the energy-based therapy. In another embodiment, the dendritic cell activating molecule is administered at least 4 days after a dose of the energy-based therapy. In another embodiment, the dendritic cell activating molecule is administered at least 5 days after a dose of the energy-based therapy. In another embodiment, the dendritic cell activating molecule is administered at least 6 days after a dose of the energy-based therapy. In another embodiment, the dendritic cell activating molecule is administered at least 7 days after a dose of the energy-based therapy. In another embodiment, the dendritic cell activating molecule is administered at least 8 days after a dose of the energy-based therapy. In another embodiment, the dendritic cell activating molecule is administered at least 9 days after a dose of the energy-based therapy. In another embodiment, the dendritic cell activating molecule is administered at least 10 days after a dose of the energy-based therapy. In another embodiment, the dendritic cell activating molecule is administered at least 11 days after a dose of the energy-based therapy. In another embodiment, the dendritic cell activating molecule is administered at least 12 days after a dose of the energy-based therapy. In another embodiment, the dendritic cell activating molecule is administered at least 13 days after a dose of the energy-based therapy. In another embodiment, the dendritic cell activating molecule is administered at least 14 days after a dose of the energy-based therapy. In another embodiment, the dendritic cell activating molecule is administered more than 14 days after a dose of the energy-based therapy.

In one embodiment, the dendritic cell activating molecule is administered between 1 and 14 days after a dose of the energy-based therapy. In another embodiment, the dendritic cell activating molecule is administered between 2 and 14 days after a dose of the energy-based therapy. In another embodiment, the dendritic cell activating molecule is administered between 3 and 14 days after a dose of the energy-based therapy. In another embodiment, the dendritic cell activating molecule is administered between 4 and 14 days after a dose of the energy-based therapy. In another embodiment, the dendritic cell activating molecule is administered between 5 and 14 days after a dose of the energy-based therapy. In another embodiment, the dendritic cell activating molecule is administered between 6 and 14 days after a dose of the energy-based therapy. In another embodiment, the dendritic cell activating molecule is administered between 7 and 14 days after a dose of the energy-based therapy. In another embodiment, the dendritic cell activating molecule is administered between 8 and 14 days after a dose of the energy-based therapy. In another embodiment, the dendritic cell activating molecule is administered between 9 and 14 days after a dose of the energy-based therapy. In another embodiment, the dendritic cell activating molecule is administered between 10 and 14 days after a dose of the energy-based therapy. In another embodiment, the dendritic cell activating molecule is administered between 11 and 14 days after a dose of the energy-based therapy. In another embodiment, the dendritic cell activating molecule is administered between 12 and 14 days after a dose of the energy-based therapy. In another embodiment, the dendritic cell activating molecule is administered between 13 and 14 days after a dose of the energy-based therapy. In another embodiment, the dendritic cell activating molecule is administered between 1 and 10 days after a dose of the energy-based therapy. In another embodiment, the dendritic cell activating molecule is administered between 2 and 10 days after a dose of the energy-based therapy. In another embodiment, the dendritic cell activating molecule is administered between 3 and 10 days after a dose of the energy-based therapy. In another embodiment, the dendritic cell activating molecule is administered between 4 and 10 days after a dose of the energy-based therapy. In another embodiment, the dendritic cell activating molecule is administered between 5 and 10 days after a dose of the energy-based therapy. In another embodiment, the dendritic cell activating molecule is administered between 6 and 10 days after a dose of the energy-based therapy. In another embodiment, the dendritic cell activating molecule is administered between 7 and 10 days after a dose of the energy-based therapy. In another embodiment, the dendritic cell activating molecule is administered between 8 and 10 days after a dose of the energy-based therapy. In another embodiment, the dendritic cell activating molecule is administered between 9 and 10 days after a dose of the energy-based therapy. In another embodiment, the dendritic cell activating molecule is administered between 1 and 7 days after a dose of the energy-based therapy. In another embodiment, the dendritic cell activating molecule is administered between 2 and 7 days after a dose of the energy-based therapy. In another embodiment, the dendritic cell activating molecule is administered between 3 and 7 days after a dose of the energy-based therapy. In another embodiment, the dendritic cell activating molecule is administered between 4 and 7 days after a dose of the energy-based therapy. In another embodiment, the dendritic cell activating molecule is administered between 5 and 7 days after a dose of the energy-based therapy. In another embodiment, the dendritic cell activating molecule is administered between 6 and 7 days after a dose of the energy-based therapy. In another embodiment, the dendritic cell activating molecule is administered between 1 and 5 days after a dose of the energy-based therapy. In another embodiment, the dendritic cell activating molecule is administered between 2 and 5 days after a dose of the energy-based therapy. In another embodiment, the dendritic cell activating molecule is administered between 3 and 5 days after a dose of the energy-based therapy. In another embodiment, the dendritic cell activating molecule is administered between 4 and 5 days after a dose of the energy-based therapy. In

### Types of Dendritic Cell Activating Molecules

Dendritic cells play a critical role in the immune system’s ability to target and kill tumor cells, but are relatively rare in most tissues. Dendritic cell activating molecules increase the total number of dendritic cells, activate the antigen presenting function of dendritic cells, increase costimulatory molecule expression, cytokine secretion, or otherwise increase their ability to prime adaptive T-cell immunity. Dendritic cell activating molecules are useful in the methods described herein. Increasing the total number of dendritic cells or activating their immunostimulatory function by administering a dendritic cell activating molecule after radiation or energy treatment can improve the ability of an individual’s immune system to target and kill cancer cells, as described in the examples.

Cancer cells can keep dendritic cells in immature states to prevent them from acting against the cancer. Immature dendritic cells can facilitate tolerance towards cancer cells while mature dendritic cells can strongly promote anticancer immunity. Promotion of maturation of dendritic cells can result in increased apoptosis in cancer cells. In one embodiment, the dendritic cell activating molecule activates maturation of an immature dendritic cell. In one embodiment, the dendritic cell activating molecule increase expression of one or more dendritic cell costimulatory molecules selected from CD70, CD80, CD86, CD40, OX40, 4-1BBL and combinations thereof. In one embodiment, the dendritic cell activating molecule increase expression or secretion of one or more dendritic cell cytokines selected from IL-12, IL-4, IL-15, or IL-17, TNFα, and combinations thereof.

A dendritic cell activator according to the methods of this disclosure can be a pathogen-associated molecular pattern (PAMP) or a synthetic version. PAMPs are small molecules conserved within a class of microbes and include without limitation glycans, glycol-conjugations, bacterial flagellin, lipoteichoic acid, peptidoglycan, and double stranded RNA. PAMPs activate of variety of innate immune receptors, known as pattern recognition receptors, expressed in antigen presenting cells and initiate adaptive immune response attributable to B and T cells. Dendritic cells express a variety of pattern recognition receptors and are activated in response to their binding to PAMPs. Pattern recognition receptors include, without limitation, toll-like receptors, NOD-like receptors, RIG-1 receptors, MDA-5 receptors, and the STING pathway. In one embodiment, the dendritic cell activating molecule activates dendritic cell activation through a toll-like receptor, a NOD-like receptor, a RIG-1 or MDA-5 receptor, a C-type lectin receptor, or a STING pathway.

Toll-like receptors are a class of receptors that are involved in the innate immune system. They are present on dendritic cells and activation of toll-like receptors with a toll-like receptor agonist or a synthetic version results in activation of the dendritic cell. In one embodiment, the dendritic cell activating molecule activates dendritic cell activation through a toll-like receptor. In another embodiment, the dendritic cell activating molecule is a toll-like receptor agonist from the list consisting of a CpG oligonucleotide, SD-101, LFX453, imiquimod, Bacillus Calmette-Guérin (BCG), monophosphoryl lipid A, Poly ICLC, GSK1795091, and combinations thereof.

NOD-like receptors are a class of pattern recognition receptors found intracellularly in dendritic cells that bind PAMPs and play a role in the innate immune system. In one embodiment, the dendritic cell activating molecule activates dendritic cell activation through a NOD-like receptor. In another embodiment, the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof.

RIG-1 and MDA-5 receptors also recognize PAMPs. Specifically, both RIG-1 receptors and MDA-5 receptors are involved in the recognition of viruses by the innate immune system. RIG-1 receptors generally bind to single or double stranded RNA strands less than 2000 base pairs, while MDA-5 receptors generally bind to virally-derived single or double RNA strands greater than 2000 base pairs. When activated, these receptors promote interferon signaling and other responses of the innate immune system. In one embodiment, the dendritic cell activating molecule activates dendritic cell activation through a RIG-1 or MDA5 receptor. In another embodiment, the dendritic cell activating molecule is a RIG-1 or MDA-5 receptor agonist selected from the list consisting of poly(I:C), Poly(dA:dT), Poly(dG:dC), 3p-hpRNA, 5′ppp-dsRNA, and combinations thereof.

C-type lectin receptors are involved in recognition of PAMPs, particularly those derived from fungi and mycobacteria. When a PAMP binds to a C-type lectin receptor, the innate immune system is activated. In one embodiment, the dendritic cell activating molecules activates dendritic cell activation through a C-type lectin receptor. In another embodiment, the dendritic cell activating molecule is a C-type lectin receptor agonist selected from the list consisting of Beta-1,3-glucan, zymosan, heat-killed C. albicans, cord factor, and Trehalose-6,6-dibehenate, and combinations thereof.

The STING pathway is involved in innate immunity and the detection of PAMPs. Activation of the STING pathway results in expression of type I interferon. In one embodiment, the dendritic cell activating molecule activates dendritic cell activity through a STING pathway. In another embodiment, the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB 1891, E-7766, or SB11285, and combinations thereof.

Co-stimulatory molecules are cell surface molecules present on antigen presenting cells including dendritic cells that can amplify or otherwise affect the activating signals that T cells receive when they interact with an antigen/MHC complex. They can affect T-cell fate and differentiation. In one embodiment, the dendritic cell activating molecule activates dendritic cell activation through a costimulatory molecule. In one embodiment, the dendritic cell activating molecule is a costimulatory molecule agonist selected from the list consisting of a CD40 agonist, aCD80 agonist, a CD86 agonist, an OX40 agonist, and combinations thereof.

CD40 is a TNF-family receptor expressed on dendritic cells. CD40 signaling results in expression of costimulatory ligands, cytokines, enhanced antigen presentation, and trafficking to the draining lymph node. In one embodiment, the CD40 agonistic is a CD40 agonistic antibody. Examples of CD40 agonist antibodies include, but are not limited to, dacetuzumab (also known as SGN-40, Seattle Genetics), CP-870,893 (University of Pennsylvania/Hoffmann-LaRoche), ADC-1013 (Alligator Bioscience AB), 2141-v11 (Rockefeller University), APX005M (Apexigen, Inc), Chi Lob 7/4 (Cancer research UKK), BG9588 (NIAMS), CFZ533 (Novartis), PG10 (PanGenetics UK Limited), BMS-986004 (Bristol-Myer Squibbs), lucatumumab (also known as HCD122, Novartis), HCD122 (Novartis), JNJ-64457107 (Janssen Research & Development), selicrelumab (also known as RO7009789), Hoffman-La Roche), ASKP1240 (Astellas Pharma Global Development), CDX-1140, and SEA-CD40 (Seattle Genetics).

Antibodies including CD40 agonistic antibodies can be administered directly to or near the tumor being treated. In some embodiments anti-CD40 agonist antibodies can be administered at or near a tumor being treated by an energy-based or radiation-based therapyat a dose about 0.1 milligrams to about 5 milligrams. In some embodiments anti-CD40 agonist antibodies can be administered at or near a tumor being treated by an energy-based or radiation-based therapyat a dose about 0.1 milligrams to about 0.2 milligrams, about 0.1 milligrams to about 0.5 milligrams, about 0.1 milligrams to about 1 milligram, about 0.1 milligrams to about 2 milligrams, about 0.1 milligrams to about 3 milligrams, about 0.1 milligrams to about 4 milligrams, about 0.1 milligrams to about 5 milligrams, about 0.2 milligrams to about 0.5 milligrams, about 0.2 milligrams to about 1 milligram, about 0.2 milligrams to about 2 milligrams, about 0.2 milligrams to about 3 milligrams, about 0.2 milligrams to about 4 milligrams, about 0.2 milligrams to about 5 milligrams, about 0.5 milligrams to about 1 milligram, about 0.5 milligrams to about 2 milligrams, about 0.5 milligrams to about 3 milligrams, about 0.5 milligrams to about 4 milligrams, about 0.5 milligrams to about 5 milligrams, about 1 milligram to about 2 milligrams, about 1 milligram to about 3 milligrams, about 1 milligram to about 4 milligrams, about 1 milligram to about 5 milligrams, about 2 milligrams to about 3 milligrams, about 2 milligrams to about 4 milligrams, about 2 milligrams to about 5 milligrams, about 3 milligrams to about 4 milligrams, about 3 milligrams to about 5 milligrams, or about 4 milligrams to about 5 milligrams. In some embodiments anti-CD40 agonist antibodies can be administered at or near a tumor being treated by an energy-based or radiation-based therapy at a dose about 0.1 milligrams, about 0.2 milligrams, about 0.5 milligrams, about 1 milligram, about 2 milligrams, about 3 milligrams, about 4 milligrams, or about 5 milligrams. In some embodiments anti-CD40 agonist antibodies can be administered at or near a tumor being treated by an energy-based or radiation-based therapy at a dose at least about 0.1 milligrams, about 0.2 milligrams, about 0.5 milligrams, about 1 milligram, about 2 milligrams, about 3 milligrams, or about 4 milligrams. In some embodiments anti-CD40 agonist antibodies can be administered at or near a tumor being treated by an energy-based or radiation-based therapy at a dose at most about 0.2 milligrams, about 0.5 milligrams, about 1 milligram, about 2 milligrams, about 3 milligrams, about 4 milligrams, or about 5 milligrams. Individuals may be administered at anti CD40 agonistic antibodies at a dose of between 0.01 to 5 mg/kg, 0.1 to 5 mg/kg, 0.01 to 2 mg/kg, 0.01 to 5 mg/kg, 0.01 to 1 mg/kg, 0.01 to 1 mg/kg, by intravenous administration.

Dendritic cells both produce cytokines and can be activated by cytokines. Cytokines can control the maturation of immature dendritic cells and activate dendritic cells. In one embodiment, the dendritic cell activating molecule activates dendritic cell activity through a cytokine receptor. In another embodiment, the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof.

The dendritic cell activating molecule may be applied directly to the site of the tumor that received either the radiation treatment or the energy treatment. In one embodiment, the dendritic cell activating molecule is administered to a tumor being treated with the dose of the radiation therapy. In another embodiment, the dendritic cell activating molecule is administered to a tumor being treated with the dose of the energy therapy. Dendritic cell activators may also be administered systemically by intravenous or subcutaneous administration.

### Treatment of a Tumor and/or Cancer

The methods described herein are useful for treating cancers and/or tumors. In certain embodiments the tumor is a solid tumor. In certain embodiments, the cancer is a blood cancer. In an embodiment, the tumor is a prostrate tumor. In an embodiment, the tumor is a melanoma. In an embodiment, the tumor is an immunotherapy resistant tumor. In an embodiment, the tumor is an immunotherapy-resistant melanoma. In an embodiment the tumor is a metastatic cancer. In an embodiment, the tumor is a metastatic breast cancer. In an embodiment of the methods, the tumor is a tumor of the prostate, breast, nasopharynx, pharynx, lung, bone, brain, sialaden, stomach, esophagus, testes, ovary, uterus, endometrium, liver, small intestine, appendix, colon, rectum, bladder, gall bladder, pancreas, kidney, urinary bladder, cervix, vagina, vulva, prostate, thyroid or skin, head or neck, glioma or soft tissue sarcoma. In an embodiment of the methods, the tumor is a prostate cancer. In an embodiment, the tumor is a malignant neoplasm.

In one embodiment, the cancer is leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt’s lymphoma and marginal zone B cell lymphoma, Polycythemia vera Lymphoma, Hodgkin’s disease, non-Hodgkin’s disease, multiple myeloma, Waldenstrom’s macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm’s tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

Also described herein are methods using combinations of radiation and/or energy based therapies and dendritic cell activating molecules are methods of treating cancers or tumors that are resistant to checkpoint inhibitor therapies. Current checkpoint inhibitor therapies target PD-1, PD-L1, PD-L2, or CTLA4, using antibodies such as pembrolizumab, nivolumab, cemiplimab, atezolizumab, avelumab, durvalumab, ipilimumab.

Also described herein are uses of dendritic cell activating molecules in a method of treating a cancer or tumor in an induvial, wherein the individual has received a dose of radiation or energy-based therapy.

Also described herein are dendritic cell activating molecules for the manufacture of a medicament for treating a cancer or tumor in an induvial, wherein the individual has received a dose of radiation or energy-based therapy.

In one aspect described here in is a method of increasing T cell infiltration into a tumor distal to a tumor being treated in an individual, the method comprising administering to the individual a dose of a radiation therapy and a dendritic cell activating molecule, wherein the dendritic cell activating molecule is administered at least one day after the radiation therapy is administered. In certain embodiments, the dendritic cell activating molecule is an antiCD40 agonistic antibody.

In one aspect described herein is a method of increasing T cell infiltration into a tumor distal to a tumor being treated in an individual, the method comprising administering to the individual a dose of an energy-based therapy and a dendritic cell activating molecule, wherein the dendritic cell activating molecule is administered at least one day after the radiation therapy is administered. In certain embodiments, the dendritic cell activating molecule is an antiCD40 agonistic antibody. In certain embodiments, the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy.

In one aspect described herein is a method of reversing T cell exhaustion in a tumor distal to a tumor being treated in an individual, the method comprising administering to the individual a dose of a radiation therapy and a dendritic cell activating molecule, wherein the dendritic cell activating molecule is administered at least one day after the radiation therapy is administered. In certain embodiments, the dendritic cell activating molecule is an antiCD40 agonistic antibody.

In one aspect described herein is a method of reversing T cell exhaustion in a tumor distal to a tumor being treated in an individual, the method comprising administering to the individual a dose of an energy-based therapy and a dendritic cell activating molecule, wherein the dendritic cell activating molecule is administered at least one day after the radiation therapy is administered. In certain embodiments, the dendritic cell activating molecule is an antiCD40 agonistic antibody. In certain embodiments, the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy.

In an embodiment, treating a tumor by the methods described herein reduces the size or volume of the tumor by about 10%, 20%, 25%, 30%, 40%, 50% or more. In an embodiment, treating a tumor by the methods described herein reduces the size or volume of a tumor that is not the tumor treated with radiation or energy-based therapy by about 10%, 20%, 25%, 30%, 40%, 50% or more. In an embodiment, treating a tumor by the methods described herein prevents metastasis of a tumor or cancer described herein.

### Pharmaceutically Acceptable Excipients, Carriers, and Diluents

In certain embodiments the dendritic cell activating molecule of the current disclosure is included in a pharmaceutical composition comprising one or more pharmaceutically acceptable excipients, carriers, and diluents. In certain embodiments, the dendritic cell activating molecule of the current disclosure is administered suspended in a sterile solution. In certain embodiments, the solution comprises about 0.9% NaCl or about 5% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the dendritic cell activating molecule of the current disclosure is shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The dendritic cell activating molecule when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

### Numbered Embodiments

Numbered embodiment 1 comprises a method of increasing T cell infiltration into a tumor distal to a tumor being treated in an individual, the method comprising administering to the individual a dose of an energy-based therapy and a dendritic cell activating molecule, wherein the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy. Numbered embodiment 2 comprises the method of embodiment 1, wherein the dose of the energy base therapy comprises a plurality of doses of energy-based therapy. Numbered embodiment 3 comprises the method of embodiments 1 or 2, wherein the energy-based therapy is Irreversible Electroporation (IRE). Numbered embodiment 4 comprises the method of embodiments 1 or 2, wherein the energy-based therapy is microwave therapy. Numbered embodiment 5 comprises the method of embodiments 1 or 2, wherein the energy-based therapy is Low-Intensity Focused Ultrasound (LOFU Numbered embodiment 6 comprises the method of embodiments 1 or 2, wherein the energy-based therapy is High-Intensity Focused Ultrasound (HIFU). Numbered embodiment 7 comprises the method of embodiments 1 or 2, wherein the energy-based therapy is cryotherapy. Numbered embodiment 8 comprises the method of any one of embodiments 1 to 7, wherein the dendritic cell activating molecule is administered at least three days after the dose of the energy-based therapy. Numbered embodiment 9 comprises the method of any one of embodiments 1 to 7, wherein the dendritic cell activating molecule is administered at least five days after the dose of the energy-based therapy. Numbered embodiment 10 comprises the method of any one of embodiments 1 to 7, wherein the dendritic cell activating molecule is administered at least seven days after the dose of the energy-based therapy. Numbered embodiment 11 comprises the method of any one of embodiments 1 to 10, wherein the dendritic cell activating molecule activates maturation of an immature dendritic cell. Numbered embodiment 12 comprises the method of any one of embodiments 1 to 10, wherein the dendritic cell activating molecule activates dendritic cell activation through a toll-like receptor, a NOD-like receptor, a RIG-1 or MDA-5 receptor, a C-type lectin receptor, a costimulatory molecule, a cytokine receptor, or a STING pathway. Numbered embodiment 13 comprises the method of any one of embodiments 1 to 10, wherein the dendritic cell activating molecule is a toll-like receptor agonist selected from the list consisting of CpG oligonucleotide, SD-101, LFX453, imiquimod, Bacillus Calmette-Guérin (BCG), monophosphoryl lipid A, Poly ICLC, GSK1795091, and combinations thereof. Numbered embodiment 14 comprises the method of any one of embodiments 1 to 10, wherein the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof. Numbered embodiment 15 comprises the method of any one of embodiments 1 to 10, wherein the dendritic cell activating molecule is a RIG-1 or MDA-5 receptor agonist selected from the list consisting of poly(I:C), Poly(dA:dT), Poly(dG:dC), 3p-hpRNA, 5′ ppp-dsRNA, and combinations thereof. Numbered embodiment 16 comprises the method of any one of embodiments 1 to 10, wherein the dendritic cell activating molecule is a C-type lectin receptor agonist selected from the list consisting of Beta-1,3-glucan, zymosan, Heat-killed C. albicans, cord factor, and Trehalose-6,6-dibehenate, and combinations thereof. Numbered embodiment 17 comprises the method of any one of embodiments 1 to 10, wherein the dendritic cell activating molecule is a costimulatory molecule agonist selected from the list consisting of a CD40 agonist, aCD80 agonist, a CD86 agonist, an OX40 agonist, and combinations thereof. Numbered embodiment 18 comprises the method of embodiment 17, wherein the CD40 agonist is an anti-CD40 agonistic antibody. Numbered embodiment 19 comprises the method of embodiment 17, wherein the anti-CD40 agonistic antibody comprises dacetuzumab, CP-870,893, ADC-1013, 2141-v11, APX005M, Chi Lob 7/4, BG9588 (NIAMS), CFZ533, PG10, BMS-986004, lucatumumab, HCD122, JNJ-64457107, selicrelumab, ASKP1240, or SEA-CD40. Numbered embodiment 20 comprises the method of any one of embodiments 1 to 19, wherein the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof. Numbered embodiment 21 comprises the method of any one of embodiments 1 to 19, wherein the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB 1891, E-7766, or SB11285, and combinations thereof. Numbered embodiment 22 comprises the method of any one of embodiments 1 to 21, wherein the dendritic cell activating molecule is administered to a tumor being treated with the dose of the energy-based therapy.

### EXAMPLES

The following illustrative examples are representative of embodiments of compositions and methods described herein and are not meant to be limiting in any way.

### Example 1 - Concurrent Administration of Radiation and αCD40 Reduces the Efficacy of The Radiation Treatment

A non-metastatic human PSA expressing TPSA murine implanted tumor model was used to assess the effect of administering αCD40 concurrently with radiation treatment (RT). Some test groups were also treated with low intensity focused ultrasound (LOFU).

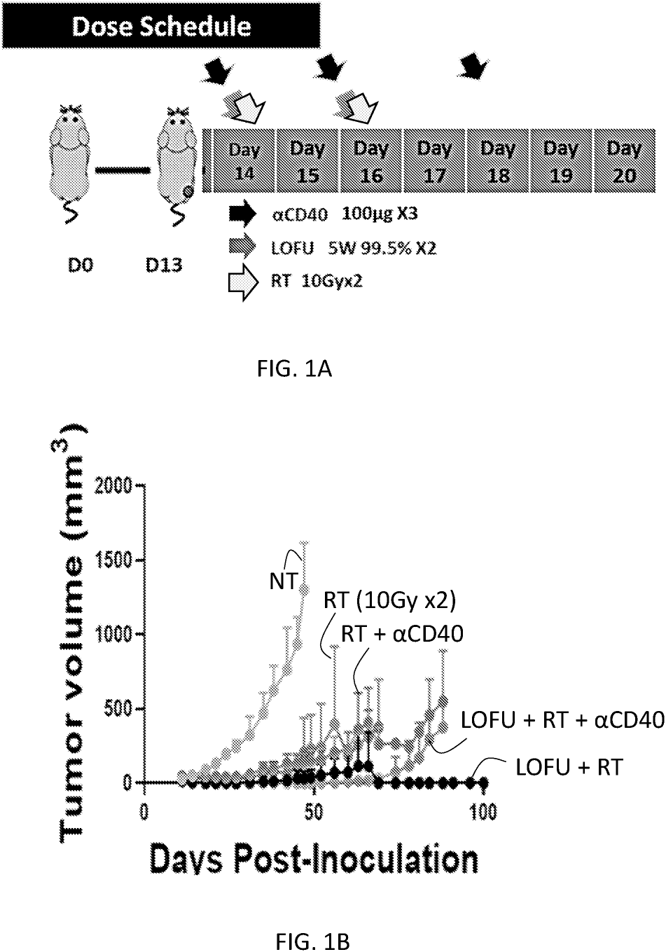

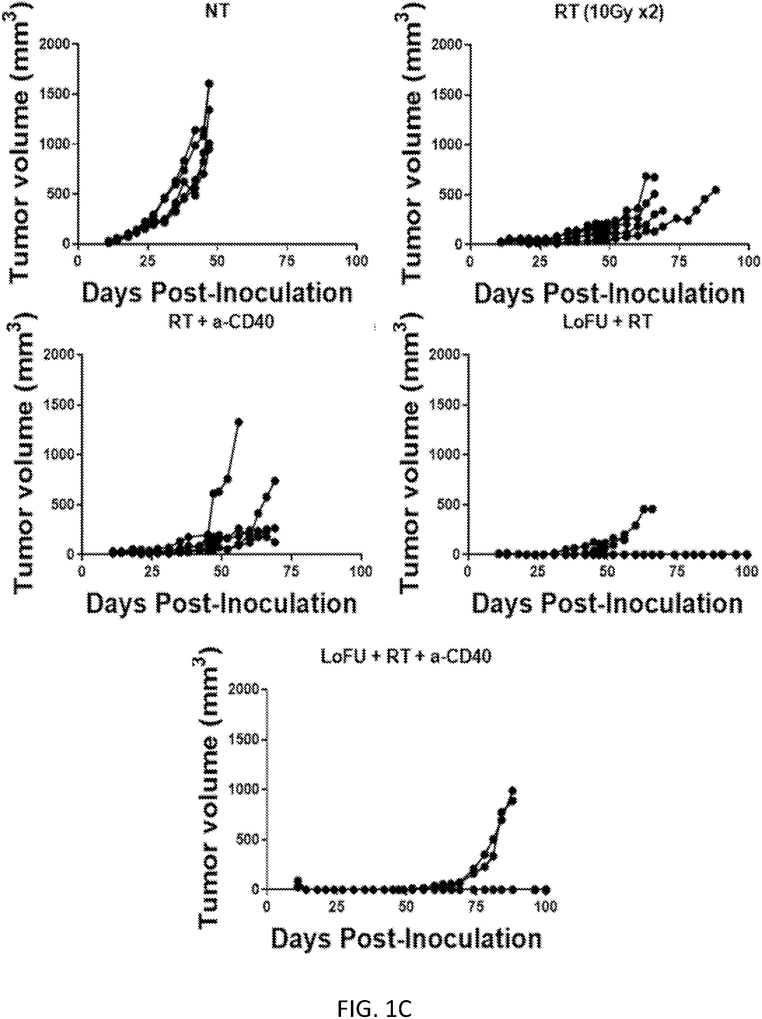

Mice were injected with 0.9×10⁶ tumor cells on the right flank. On day 14-17, mice with palpable tumors were randomly segregated into different treatment groups. The treatment groups were control (un-irradiated), RT (10Gy×2), RT (10Gy×2) + αCD40, RT (10Gy×2)+LOFU and LOFU+RT+αCD40. Mice were treated with RT and LOFU (5W 99.5%) on day 14 and day 16 with concurrent αCD40 therapy (day 14, day 16, and day 18; 3×100µg/ per mice), as depicted in FIG. 1A. Tumors were measured every 3-4 days. For tumor measurements, perpendicular tumor diameters were measured using a digital caliper and tumor sizes were calculated as lxbxhx3.14/6; where 1 is the longest dimension of tumor, while b and h are other two perpendicular dimensions.

FIG. 1B depicts the average tumor volume for each treatment over the first 100 days, while FIG. 1C depicts the tumor volume over the first 100 days in each individual mouse. In all irradiated mice, tumor growth was significantly reduced or completely regressed at 25 days post tumor injection, as depicted in FIG. 1B. However, most mice regrew tumors at the primary site. When the RT and RT+LOFU groups are compared with αCD40+ RT and RT+LOFU treated animals, it was found that αCD40 reduced the efficacy of the radiotherapy.

### Example 2 -Administering αCD40 Concurrently With Radiation Treatment in PSA Transgenic Mice

In this example, the effect of administering αCD40 concurrently with radiation treatment (RT) in PSA transgenic mice was assessed. Some test groups were also treated with low intensity focused ultrasound (LOFU).

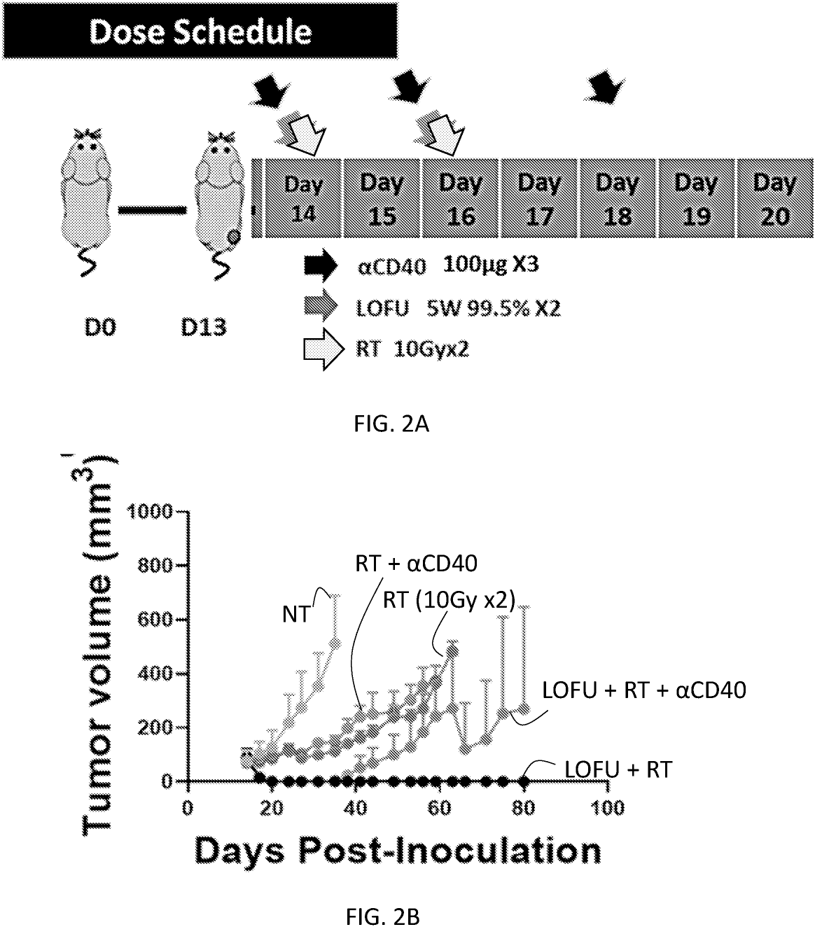

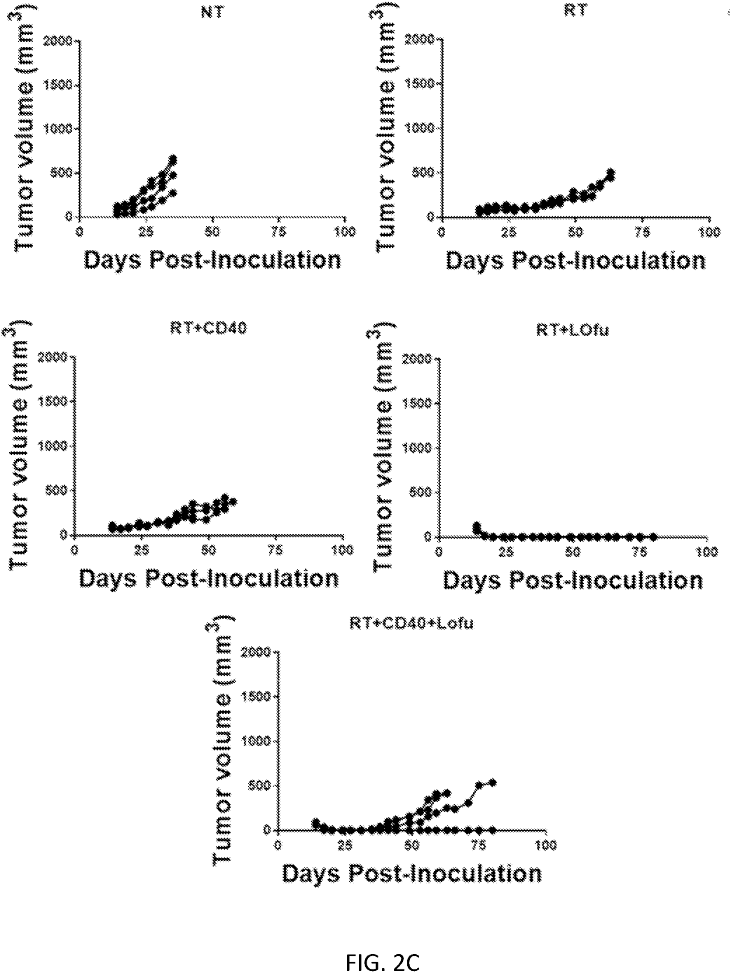

The experimental treatment is depicted in FIG. 2A. PSA transgenic mice, which lack PSA-specific CD8 cells, were injected with 0.9×10⁶ tumor cells on the right flank. On day 14-17, mice with palpable tumors were randomly segregated into different groups as control (un-irradiated), RT (10Gy×2), RT (10Gy×2) + αCD40, RT (10Gy×2)+LOFU and LOFU+RT+αCD40. Mice were treated with RT and LOFU (5W 99.5%) on day 14 and day 16 with concurrent αCD40 therapy (day 14, day 16, and day 18; 3×100µg/ per mice). Tumors were measured at every 3-4 days.

The growth of tumor volume per treatment is depicted in FIG. 2B, while FIG. 2C illustrates tumor growth in individual mice. In the PSA transgenic mice, concurrent administration of αCD40 led to significant tumor growth compared to the LOFU and RT treated group (p<0.05). When the RT+LOFU groups were compared with αCD40+ RT and RT+LOFU treated animals, it was found that αCD40 reduced the efficacy of the radiotherapy (p>0.05).

### Example 3 -Administering αCD40 Post-Ablation Enhances the Local and Systemic Efficacy Of Radiotherapy in a Checkpoint Blockade (αCTLA4) Resistant Tumor

This example assessed the effect of treating immunotherapy resistant melanoma cells with αCD40 administered after radiation treatment consisting of ionizing radiation (IR).

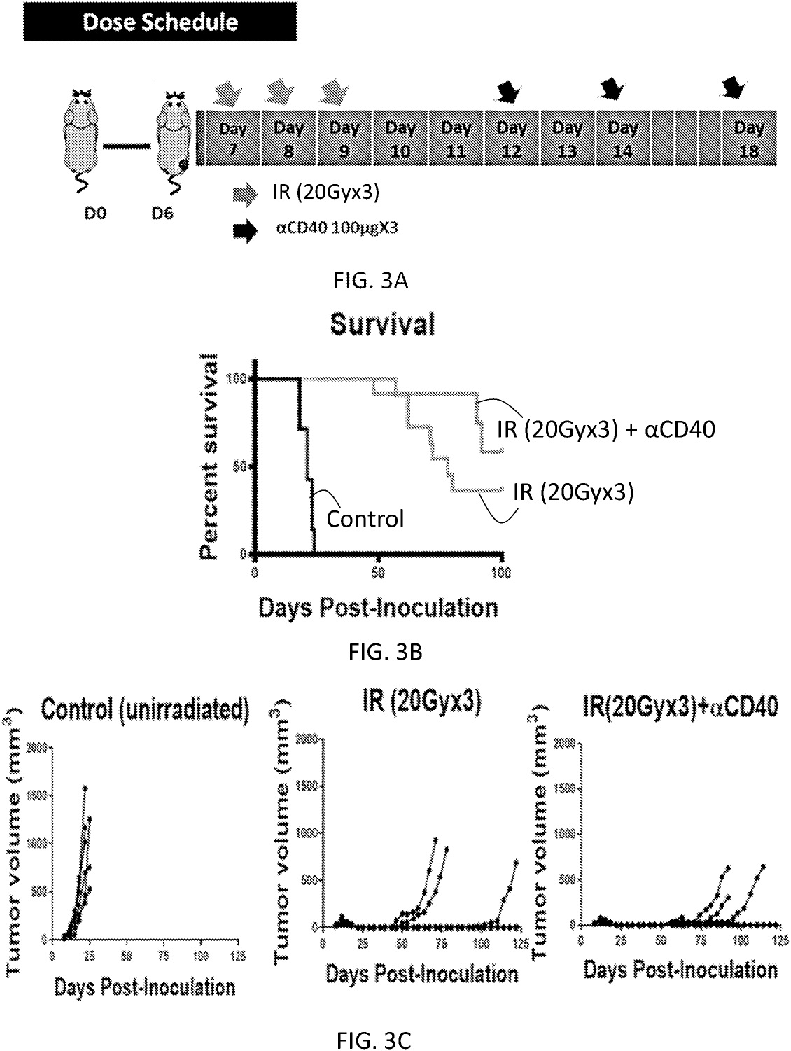

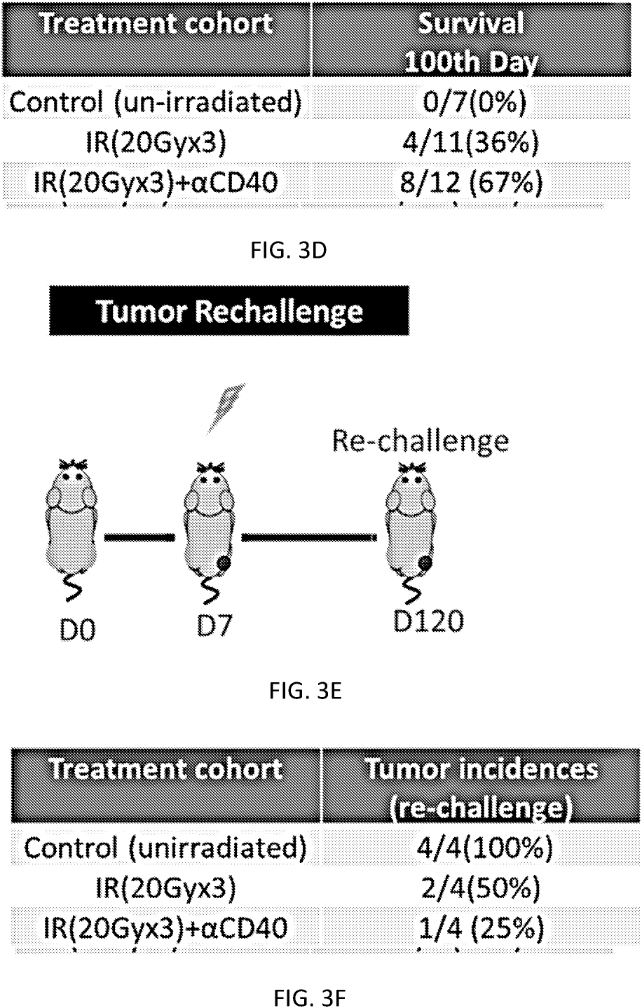

The treatment schedule of the mice is depicted in FIG. 3A. Mice were injected subcutaneously with 0.2x10⁶ RES499 immunotherapy (αCTLA-4) resistant murine melanoma cells in the right flank. 7 days post injection, mice were randomly segregated into different treatment groups as control (un-irradiated), IR (20Gyx3) and IR (20Gyx3) + αCD40. Mice were irradiated with 3 fractions (1 fraction every day) of 20 Gy at 7, 8, and 9 days post injection. αCD40 (3x100ug) was administered at 12, 14, and 18 days post injection.

When administered sequentially, αCD40 effectively enhanced the long-term survival and cure in the RES499 tumor bearing mice. As seen in FIG. 3B, all untreated mice died before 50 days post tumor injection. At 100 days post tumor injection, less than 50% of the mice treated with radiation alone survived. Over 50% of the mice that had received radiation treatment followed by αCD40 treatment were alive at day 100. Furthermore, in all the irradiated mice, tumor growth was significantly reduced or completely regressed at 25 days post tumor injections, as seen in FIG. 3C. However, most of the mice regrew tumors at the primary site. At 100 days post injection, irradiated mice which had been treated with αCD40 had higher survival rates than mice that had only been treated with radiation. 67% of the mice in the IR (20Gyx3) +αCD40 group were tumor-free on Day 90 compared with the 36% in the IR group, as seen in FIG. 3D.

At day 120, the tumor-free mice were re-challenged with RES499 cells, as depicted in FIG. 3E. Tumor incidences after re-challenge varied based on initial treatment. While age matched untreated mice showed 100% incidence by day 7, mice treated with radiation alone and mice that received αCD40 treatment subsequent to radiation showed 50% and 25% incidence respectively on day 25 post tumor re-challenge, as depicted in FIG. 3F.

This example showed that treatment with αCD40 enhanced the radiotherapy associated survival and cure in mice with immunotherapy resistant tumors. The re-challenge experiment showed an increased adaptive memory response against immunotherapy resistant tumors.

### Example 4 -Radiotherapy in Combination With Sequential αCD40 Reduces the Growth of Abscopal RES499 Melanoma Tumors

This example assessed the ability of αCD40 administration following radiation (IR) to retard the abscopal tumor growth of tumors resistant to radiotherapy and αCTLA-4 therapy.

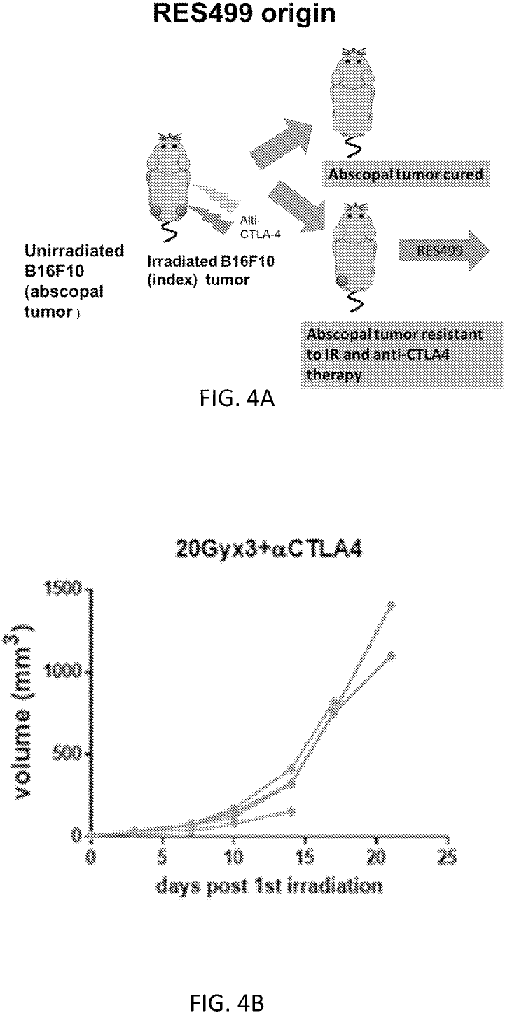

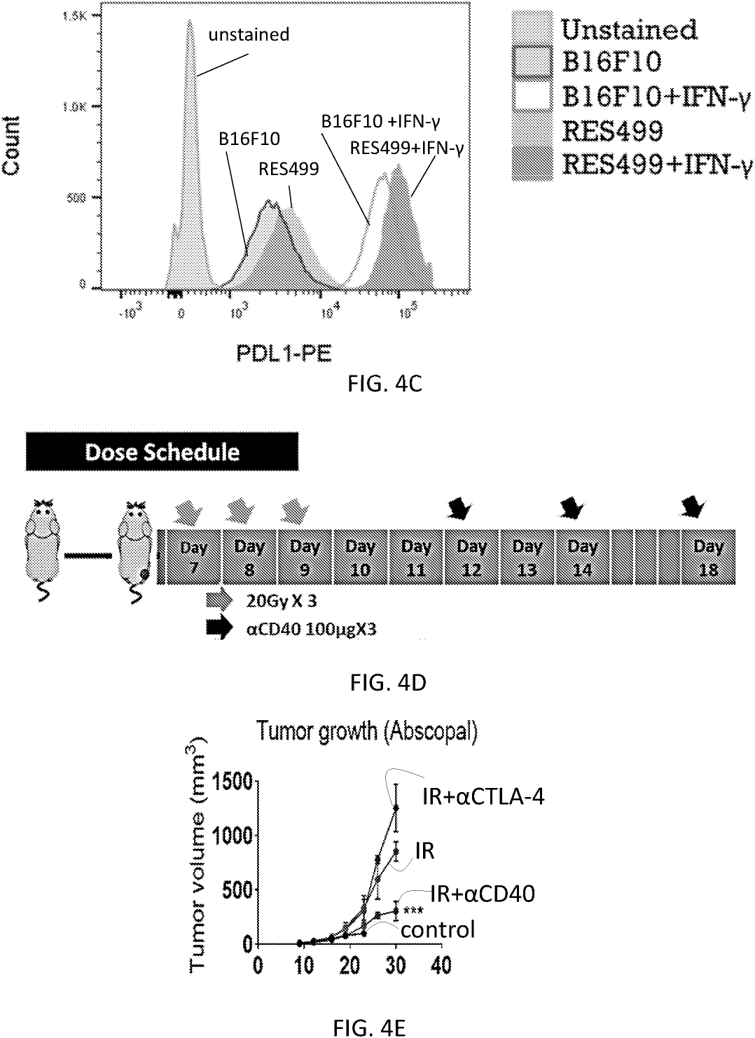

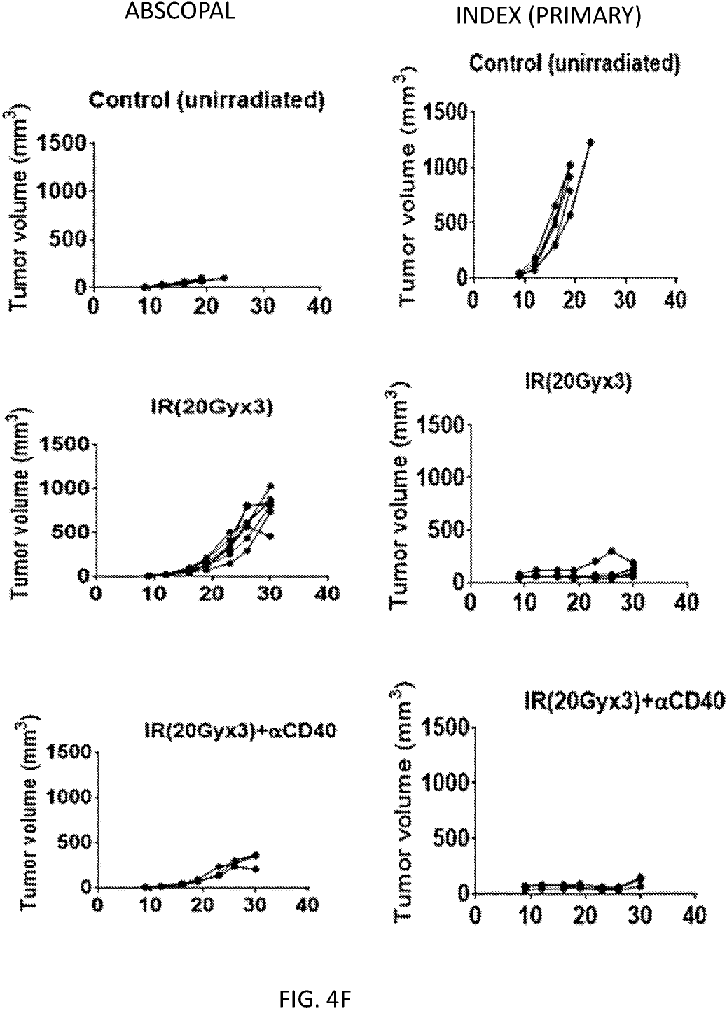

The RES499 tumor line was developed from tumors which were non-responsive to the systemic effects of combined radiotherapy and αCTLA-4 therapy, as depicted in FIG. 4A. These cells were resistant to IR and αCTLA-4 therapy, as depicted in FIG. 4B, where tumor size rapidly increased in mice which receive both radiation and αCTLA-4 therapy. The resistance of these cells was due to elevated IFNγ signaling. As shown in FIG. 4C, elevated IFNγ signaling in these cells resulted in increased expression of PDL1 in the tumor cells.

C57BL/6 mice were injected subcutaneously with 0.2×10⁶ RES499 melanoma cells in the right flank (index tumor; irradiated) on day 0 and 0.1×106 RES499 cells in the left flank (abscopal tumor; non-irradiated) on day 4. On days 7-9, when primary tumors were palpable, animals were randomly assigned to the different treatment groups. For treatment, mice were irradiated with 3 fractions (1 fraction every day) of 20 Gy each from day 7-9. αCD40 (3×100ug) was administered on day 12, day 14, and day 18, as depicted in FIG. 4D.

FIG. 4E shows the effect of treatments on mean tumor volume in the abscopal tumor. Mice which received both radiation treatment and αCD40 treatment had a much lower rate of tumor growth than mice which received radiation alone or radiation in conjunction with αCTLA-4 treatment.

FIG. 4F shows the total tumor growth of the index (primary) tumor in both treated and control mice over 30 days. In response to the ablative radiation dose, the primary index tumor growth was reduced in all the irradiated mice when compared to untreated tumor growth (p<0.0001). In mice that received radiation alone, the abscopal tumors showed a large amount of tumor growth. However, mice that received a combination of IR and αCD40 treatment had a significant reduction in the growth of the abscopal tumors (p<0.001). On day 30, abscopal tumor growth in mice treated with both IR and αCD40 was reduced by up to 64% (p<0.0001) compared to the mice treated with IR alone.

This experiment showed that a combination of IR and αCD40 treatment significantly reduced the growth of both primary and abscopal tumors in an immunotherapy-resistant tumor line.

### Example 5 - αCD40 Induces Co-stimulatory Molecules and Type 1 Inflammation in CD103⁺ Dendritic Cells in Tumors

This example assessed the effect of the systemic αCD40 therapy in combination with radiation (IR) on tumor-infiltrating host cells.

Three days after the second dose of αCD40, tumors were excised and digested postmortem using a cocktail of collagenase type IV and DNase. After digestion at 37° C. for 30 minutes, cells were passed through a 70-µm filter. Cells were stained for cell surface and cytosolic proteins. Cells were then analyzed by flow cytometry and zombie IR (Thermo Fisher) was used as a viability dye.

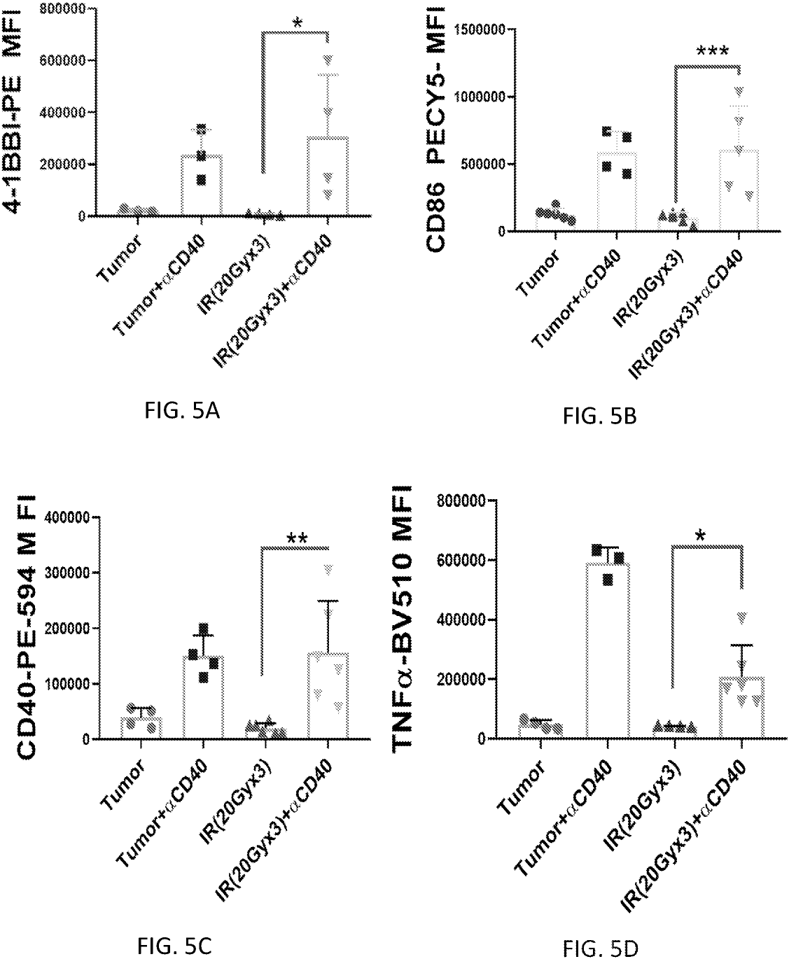

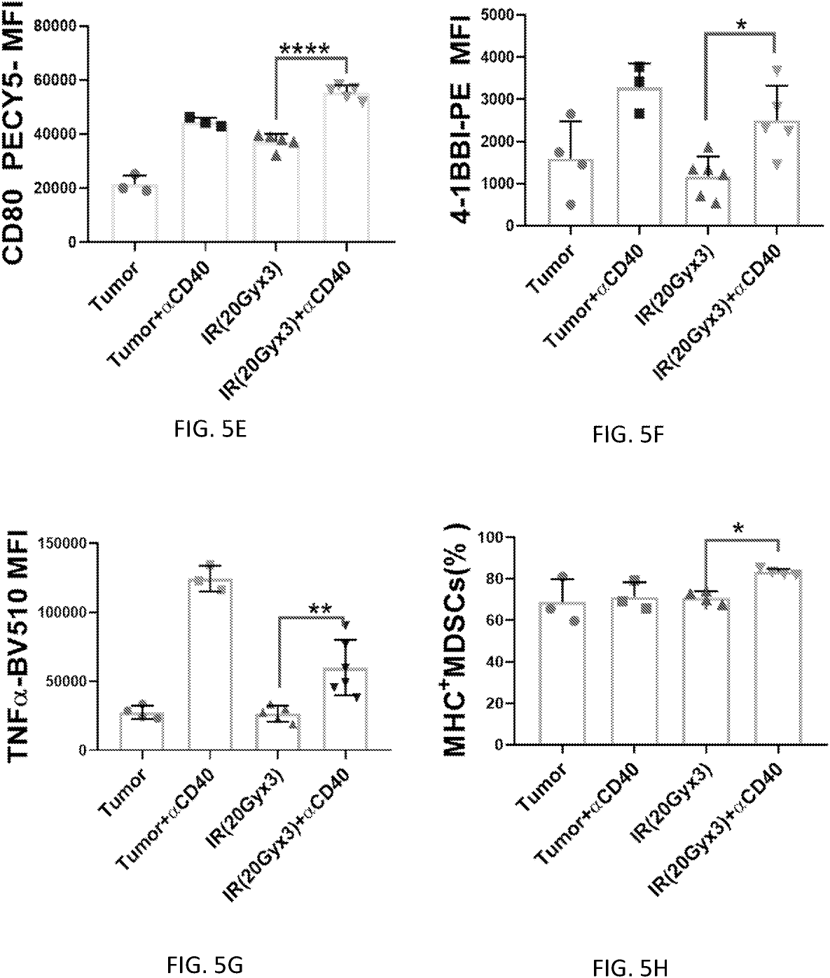

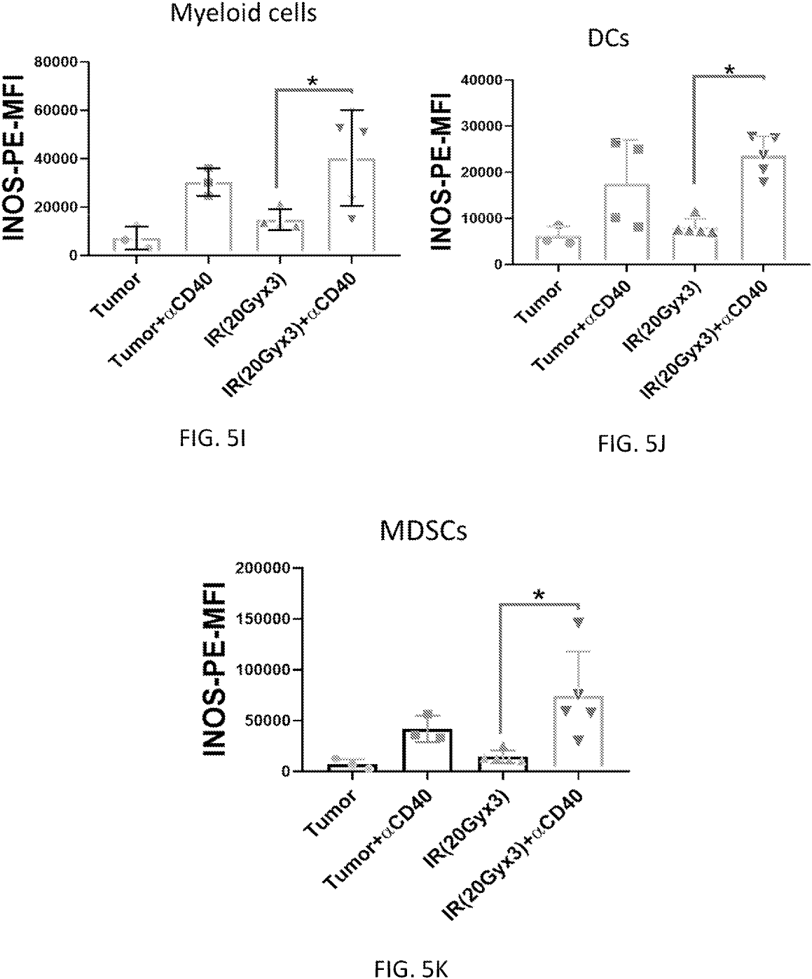

There was a significant increase (p<0.5) in the co-stimulatory markers (4-1BBL, CD40 and CD86) and type 1 inflammation (TNF-α) in the tumor infiltrating CD103⁺ dendritic cells (DC) derived from mice that had received a combination of αCD40 and IR when compared to the IR treated group, as depicted in FIGS. 5A-5D.

The agonist CD40 antibody also affected the immature suppressor cells of myeloid origin (Ly6C high CD1lb⁺). The myeloid derived suppressor cells (MDSC) showed an increase in co-stimulatory markers CD80 and 4-1BBL when derived from mice treated with both radiation and αCD40, as depicted in FIGS. 5E-5F, compared to mice treated with radiation alone. Furthermore, treatment with both radiation and αCD40 also resulted in an increase in type 1 inflammation markers in the MDSCs, as illustrated by the increased levels of TNFα in FIG. 5G. Finally, these mice also showed an increase in antigen presentation, demonstrated by the increased percentage of MHC⁺ MDSCs depicted in FIG. 5H.

Inducible nitric oxide synthetase (INOS) is a cell-killing effector of the myeloid and DCs. Treatment with αCD40 significantly increased INOS levels in the CD103⁺DCs, MDSCs, and total pool of the myeloid cells, compared to treatment with radiation alone (FIGS. 5I-5K). The increase in the cytosolic levels of NOS suggested increased tumor killing functions of the innate host cells.

### Example 6 - αCD40 Induces Co-stimulatory Molecules and Down Regulates Immune Suppressive Functions in Draining Lymph Node

This example assessed the effect of the systemic αCD40 therapy in combination with radiation (IR) on infiltrating host cells in the draining lymph node (DLN).

Three days after the second dose of αCD40, DLNs were harvested and cells were passed through a 40-µm filter. Cells were stained for cell surface and cytosolic proteins. Cells were then analyzed by flow cytometry and zombie IR (Thermo Fisher) was used as a viability dye.

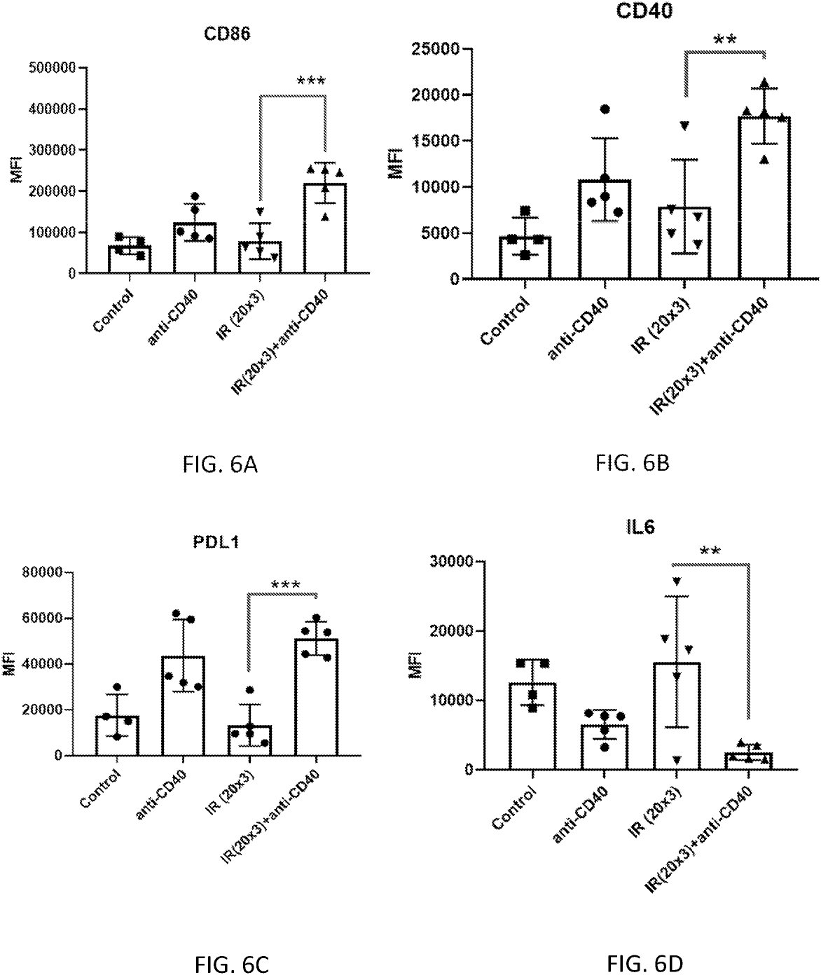

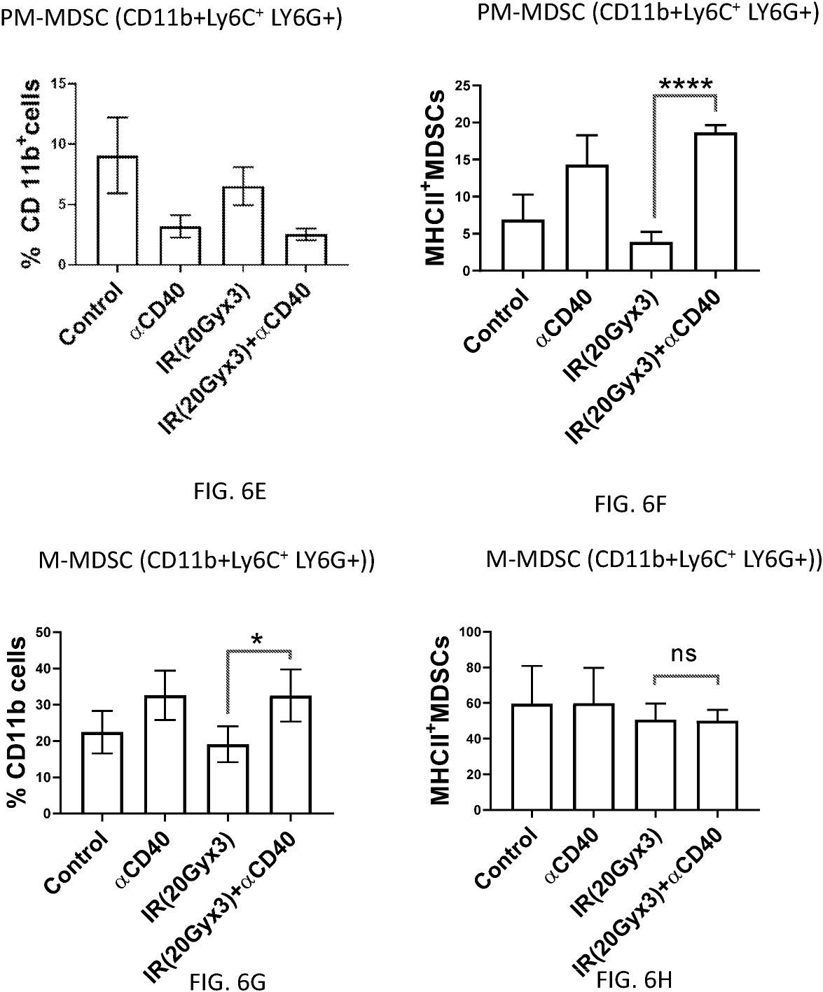

In the gross CD11b⁺ leucocytes and its subpopulations, activation associated costimulatory molecules CD86 and CD40 were increased (p<0.01-0.001), as depicted in FIGS. 6A-6B. PDL1 levels were increased in mice that had received αCD40 treatment compared to untreated mice, and significantly increased between irradiated mice and mice that received both radiation and αCD40 treatment, as seen in FIG. 6C. Furthermore, αCD40 treatment also affected the immune suppressive function in the DLN. IL6 levels in the CD11b⁺ cells were significantly decreased in mice treated with radiation and αCD40 compared to mice treated with radiation alone (FIG. 6D, p<0.01). IL6 signaling is a critical in driving the immunosuppressive effects of the radiation.

The granulocytic MSDCs (PM-MDSCs) showed a decrease in the percent of CD11b⁺ cells in the DLN after treatment with both radiation and αCD40, depicted in FIG. 6E. These cells also showed an increase in antigen presenting ability (p<0.0001) when compared to the group treated with radiation alone, as depicted in FIG. 6F. Furthermore, there was an increased infiltration of the MHCII high myeloid MDSCs in mice that had been treated with both αCD40 and radiation compared to mice that received radiation alone (FIGS. 6G-6H). Results suggest that while combination treatment promoted the activation and functional competence of the DCs and myeloid cells, immature and suppressive suppressor cells were switched to their activated and antigen presenting states.

### Example 7 - Combined αCD40 and the IR Treatment Enhance the CD8 Effector Function in Abscopal Tumor

This example assessed the effect of sequential αCD40 treatment on CD8 effector function.

Characterization of T cells in the αCD40 +IR treated lungs showed that there was an increase in the frequency and the functional competence of the effector cytotoxic CD8 T cells when compared with the IR alone treated group.

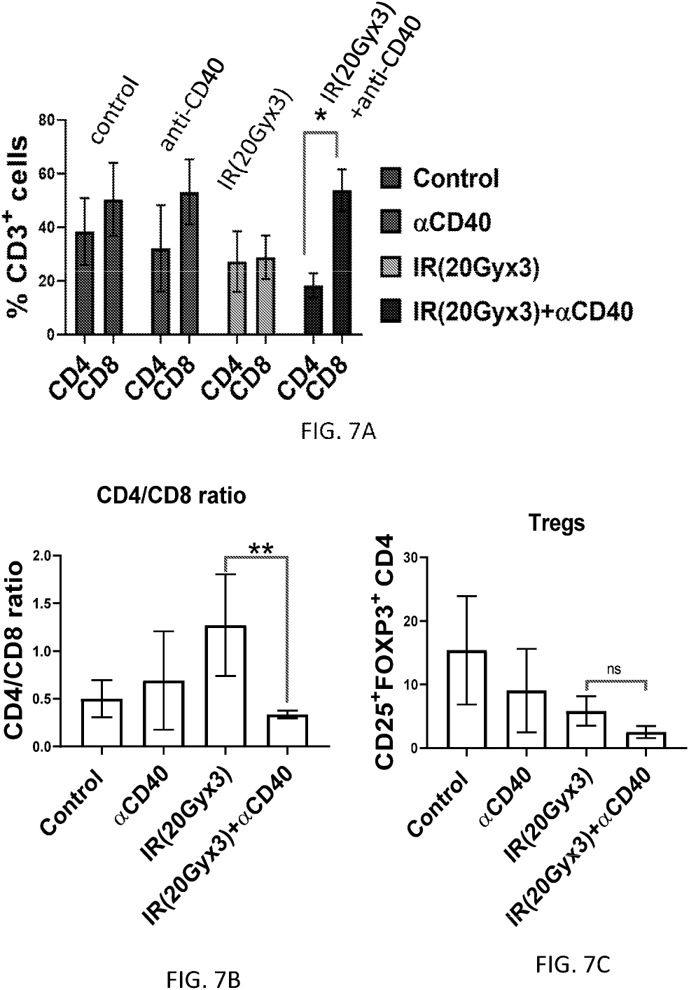

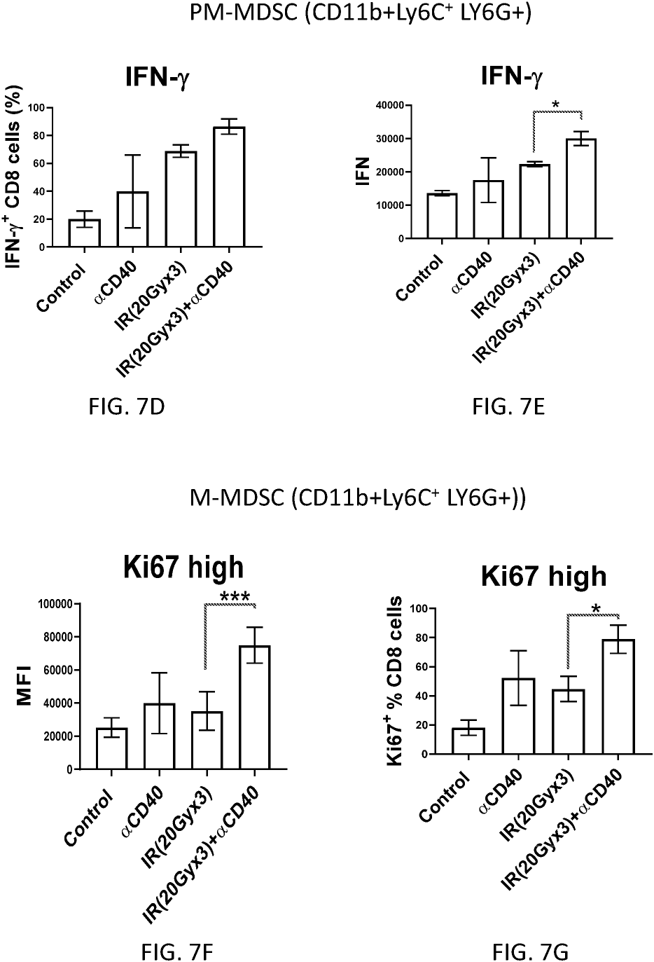

The CD8 proportion in the tumor was assessed by measuring the frequency of CD8 cells and CD4/CD8 ratio which is a marker of an effective anti-tumor immune response. αCD40 treatment affected the CD8 proportion in the tumor. There was a significant decrease in CD8 numbers in the tumor derived from mice that had received a combination treatment when compared to mice that had received radiation alone. This was both an increased seen as both an increase in CD8 frequency as well as in a reduced CD4/CD8 ratio (FIGS. 7A-7B, p<0.01). Furthermore, a decrease in the regulatory T cell proportion in the CD4 helper cells was also seen in the tumors from mice that had received both αCD40 treatment and radiation when compared to mice that had received only radiation, as depicted in FIG. 7C.

Functions of CD8 cells were assessed using both the frequency of the functional IFNγ⁺ cells and the increased proliferating cells. αCD40 treatment increased both the percent of IFNγ⁺ CD8 cells and the mean fluorescent intensity (MFI) of IFNγ⁺ CD8 cells, as depicted in FIGS. 7D-7E. This increase occurred both when comparing unirradiated mice and when comparing irradiated mice. Mice which received radiation and αCD40 treatment had both the greatest percent of IFNγ⁺ CD8 cells and the highest MFI of IFNγ⁺ cells. Furthermore, as depicted in FIGS. 7F-7G, αCD40 administration following radiation increased the proportion of the Ki67⁺ high cells indicative of the highly proliferating CD8 cells. The increase in the frequency of IFNγ⁺ CD8 and the proliferation of IFNγ⁺ CD8 cells demonstrates that the myeloid activation in the tumor was associated with the concurrent increase in the functional CD8 cells.

### Example 8 - Combined αCD40 and the IR Treatment Enhance the CD8 Effector Function in the Draining Lymph Node

This example assessed the effect of αCD40 treatment combined with radiation on CD8 effector function in the abscopal tumor draining lymph node (DLN).

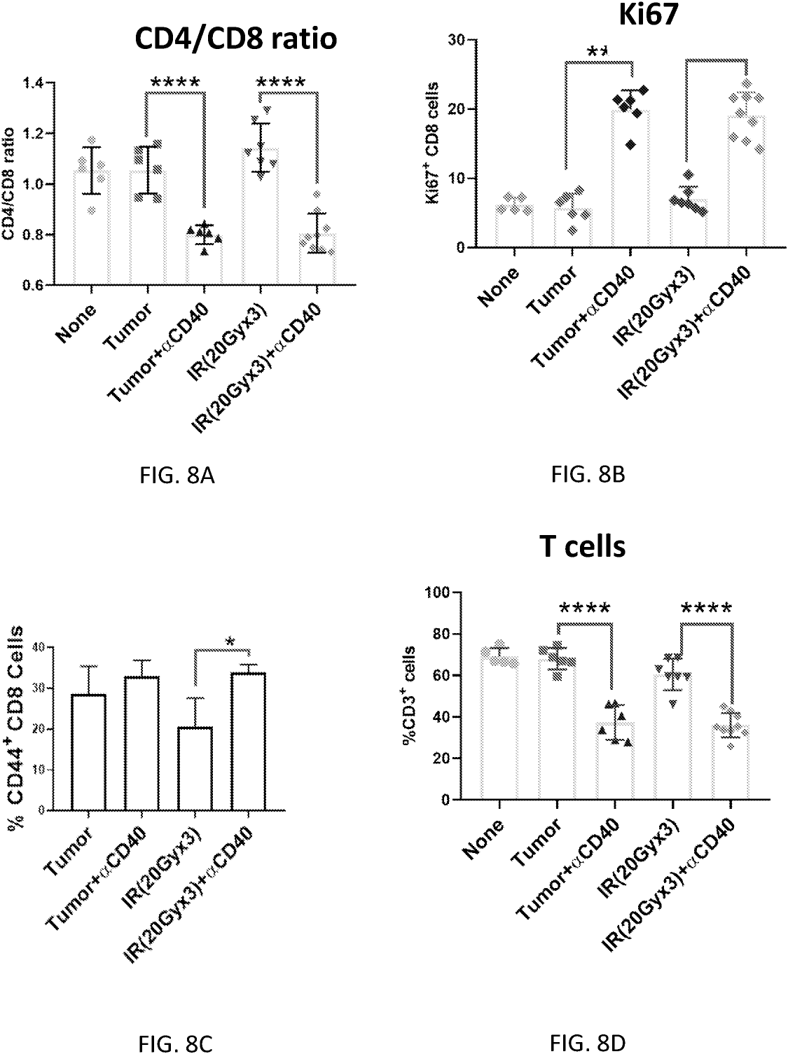

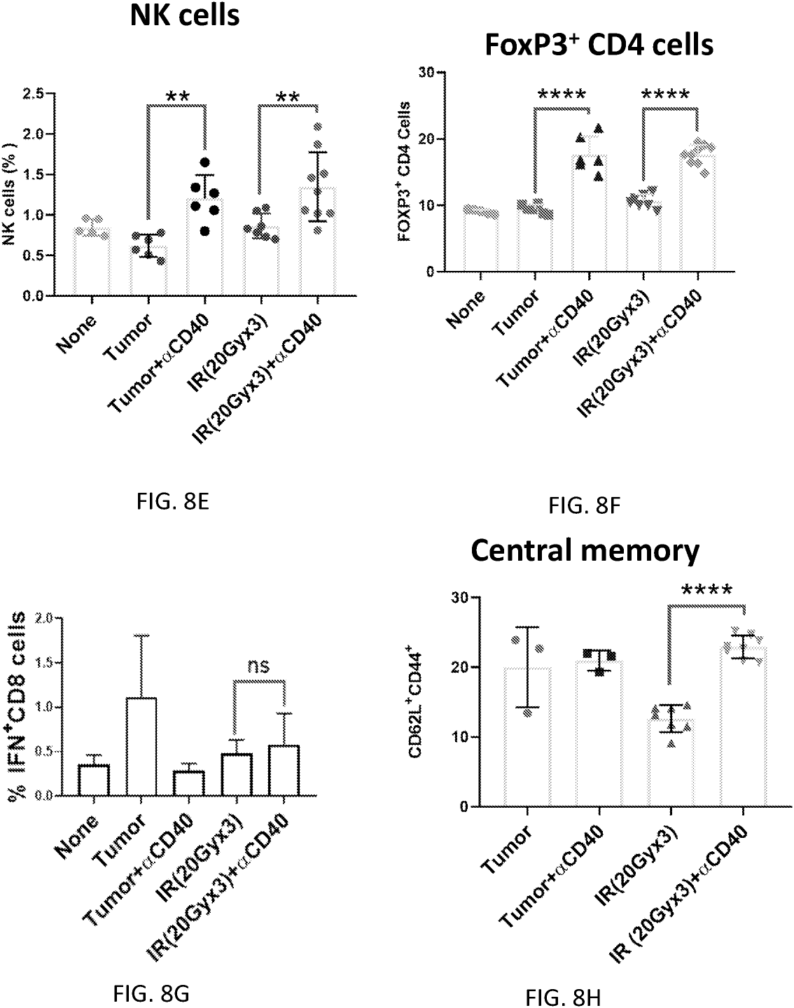

The effect of αCD40 and radiation treatment on CD8 effector function in mice was measured in the abscopal tumor DLN. The CD4/CD8 ratio was decreased in mice that had received both αCD40 and radiation, compared to mice that had received radiation alone, as illustrated in FIG. 8A.

This was due to the highly activated state of the cells, which was determined by both the increase in Ki67⁺ cells and CD44⁺ cells depicted in FIGS. 8B-8C. Furthermore, an increase in the infiltration of the DLN of the CD8 and natural killer (NK) cells depicted in FIGS. 8D-8E was also suggestive of the development of an effective anti-tumor immunity. The T cell proportion in the CD45⁺pool of the DLN compartment was significantly reduced in the mice which received αCD40 treatment and radiation treatment, compared to those that received radiation alone (p<0.0001, FIG. 8D). An increased CD8⁺proportion was suggestive of the increased effector CD8 function. This was further strengthened by the increase in IFNγ⁺ cells, depicted in FIG. 8G, and the significant increase in the proliferating Ki67⁺ CD8 cells (p<0.01), depicted in FIG. 8B. Furthermore, an increase in the Ki67 high CD8⁺ cells in the DLN was suggestive of efficient antigen presentation. Activation of FOXP3⁺ CD4 cells increased significantly in the group that received both αCD40 treatment and radiation, compared to the group that received radiation alone, as depicted in FIG. 8F. There was also a significant increase in FOXP3⁺ cells when comparing the tumor cells to the tumor cells that had received αCD40 treatment. Mice that had received both αCD40 and radiation treatment showed an increase in CD62L⁺ CD44⁺ cells, indicating an increase in central memory, as depicted in FIG. 8H. An increase in the CD8 T cell functions in the IR+αCD40 group compared to the IR group suggested that myeloid activation through CD40 agonism was translated into effective antitumor immune functions through enhanced CD8 proliferation and competency.

### Example 9 -Sequential Administration of αCD40 Post IR Ablation Inhibits the Metastatic Disease and Associated Death in Tumor Bearing Mice

This example assessed the effects of αCD40 administration combined with radiation treatment of metastatic cancer using the murine orthotropic breast tumor cell line 4T1. Radiation treatments include ionizing radiation (IR), and post ablation modulation (PAM, 4 doses of 0.5 Gy IR dose every day).

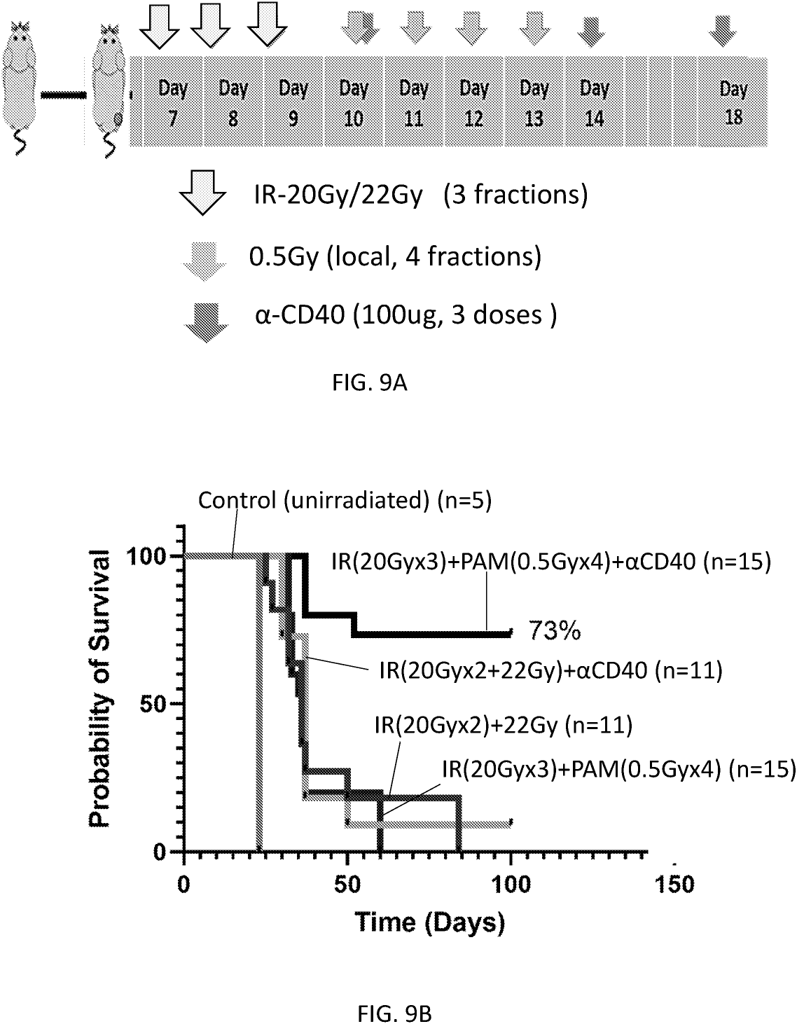

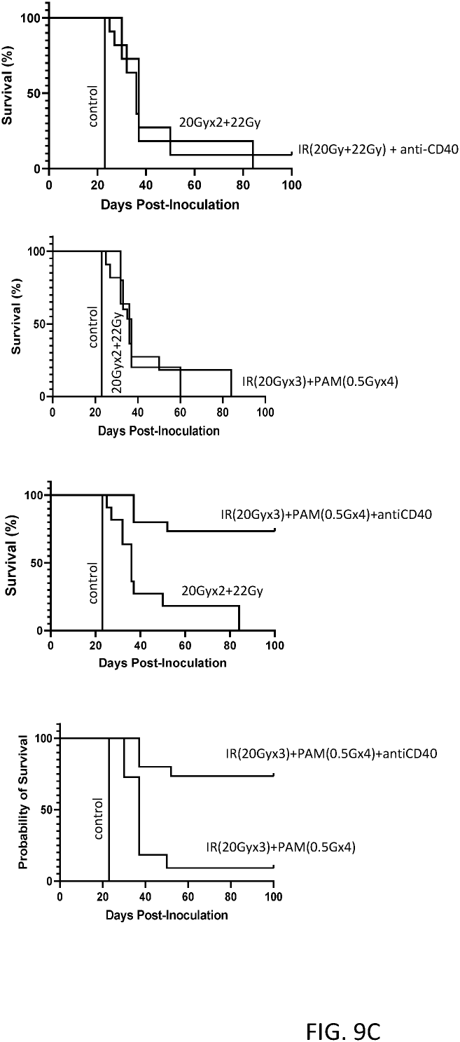

0.2×10⁶ 4T1 cells were injected in the mammary fat pad of BALB/c mice (syngeneic to 4T1). At day 7, mice with palpable tumors were randomly segregated into 5 groups: control (un-irradiated), IR (20Gy×3) + PAM (0.5Gy×4), and IR (20Gy×3) + PAM+ αCD40. Mice were irradiated on day 7-9 and αCD40 was given post IR (Day 10, 14 and 18). 0.5Gy×4 doses (PAM) were given on days 10-13. Tumor volumes and survival was recorded at multiple times. The treatment protocol is depicted in FIG. 9A.

When combined with radiotherapy, αCD40 significantly inhibited the metastatic events and improved overall mouse survival, as seen in FIGS. 9B-9C. 73% of the 15 mice in the IR+PAM+ αCD40 group survived to 100 days post tumor cell injection. All unirradiated mice died before 40 days post tumor cell injection.

This example showed that sequential treatment with radiation and αCD40 can effectively treat metastatic disease and inhibit death in a metastatic model.

### Example 10 -Treatment of Cancer With Post Ablation Modulation (PAM) and Additional Therapies

This example assessed the effects of αCD40 administration combined with radiation treatment on abscopal (non -irradiated) tumor growth using the murine melanoma lines B16F10 and RES499 (checkpoint resistant line). Radiation treatments include ionizing radiation (IR).

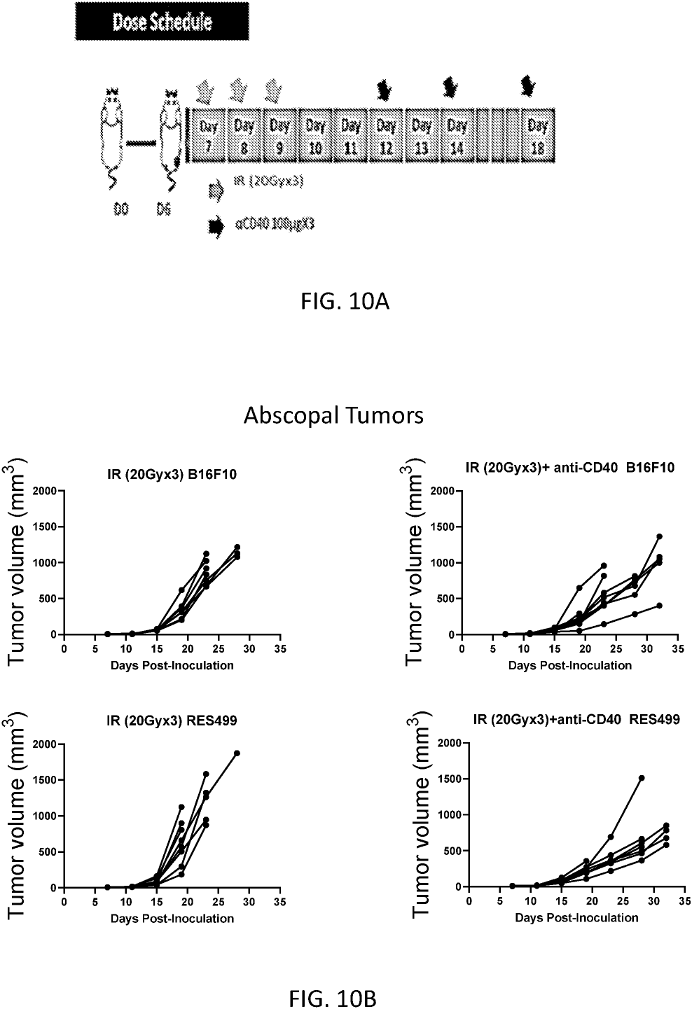

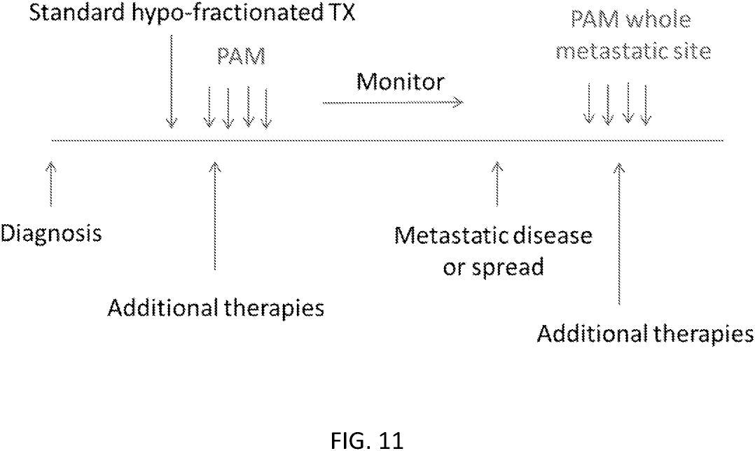

C57BL/6 mice were injected subcutaneously with 0.2×10⁶ RES499 and B16 melanoma cells in the right flank (index tumor; irradiated) on day 0 and 0.1×106 RES499 cells in the left flank (abscopal tumor; non-irradiated) on day 4. On days 7-9, when primary tumors were palpable, animals were randomly assigned to the different treatment groups. For treatment, mice were irradiated with 3 fractions (1 fraction every day) of 20 Gy each from day 7-9. αCD40 (3×100ug) was administered on day 12, day 14, and day 18, as depicted in FIG. 10A. Tumor volumes of index and the primary tumor and survival were also recorded at multiple times. The treatment protocol is depicted in FIG. 10A.

Mice treated with αCD40 showed lower tumor volumes and higher rates of survival than mice treated with radiation alone (FIG. 10B). This effect was seen both in the murine melanoma model using B16F10 (FIG. 10B, top panels) and in the checkpoint-resistant line murine melanoma model using RES499 (FIG. 10B, lower panels).

This example showed that sequential treatment with radiation and αCD40 therapy can effectively inhibit tumor growth in a melanoma model and in checkpoint resistant tumors.

### Example 11 -Treatment of Cancer With Post Ablation Modulation (PAM) and Additional Therapies

Patients with cancer may follow the disease and treatment progression shown in FIG. 11. A patient is diagnosed with cancer. The patient is treated with a standard hypo-fractionated therapy, followed by a combination of treatment with PAM and additional therapies. The additional therapy may be αCD40 therapy. The patient is then monitored. If metastatic disease occurs, the whole metastatic site is treated with PAM and additional therapies. This may improve survival compared to conventional methods of treatment.

### Example 12 - Anti-CD40 Therapy Reverses the Exhaustion of the Tumor-Infiltrating T Cells (PD1int EomesS low ) to GrBZ+Ki67+ Subset in Mice Treated With or Without Irradiation

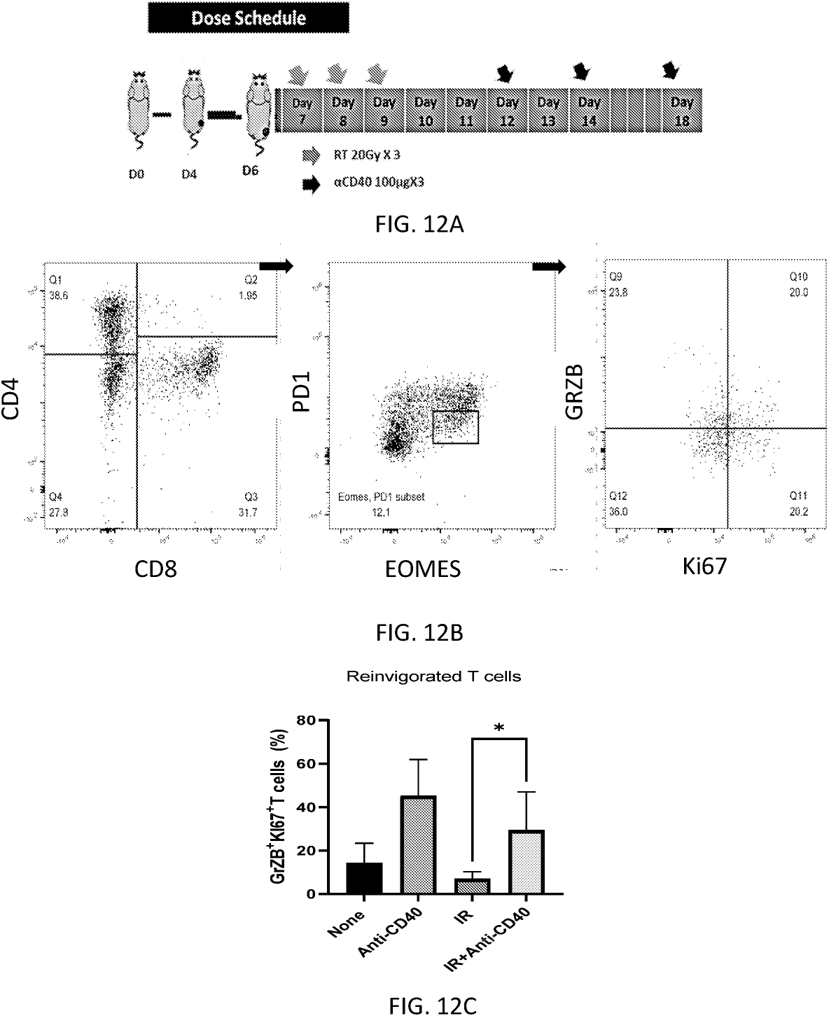

This example assessed the effect of anti-CD40 therapy on the exhaustion of tumor-infiltrating cells. The experimental protocol is depicted in FIG. 12A. C57BL/6 mice were injected s.c. with 0.2×106 RES499 melanoma cells in the right flank (index tumor; irradiated) on day 0 and in the left flank (abscopal tumor; non-irradiated) on day 4. On day 7-9, when primary tumors were palpable, animals were randomly assigned to the different treatment groups. For treatment, mice were irradiated with 3 fractions (1 fraction every day) of 20 Gy each from day 7-9. αCD40 (3×100ug) was administered on at D12, D14 and D18.

IR has been shown to induce exhaustion of T cells during the radiotherapy. IR alone group showed minimal population of the functional subtype (GrB+KI67high) in the early exhausted cells (PDlintEomeshi). In the IR+anti-CD40 group, functional subtype of the exhausted population was significantly increased (p<0.05). Early exhaustion is marked by the PD1 intermediate and EOMES low CD8 cells (PDlintEomeshi). Anti-CD40 +IR combination group increased the Ki67 (proliferating) high GRZ+ (granzyme secreting) population in the pool suggesting a reversal of the exhausted phenotype (FIGS. 12B-12C).

### Example 13 - Depletion of Immune Cells in Mediating anti-CD40 and IR Treatment

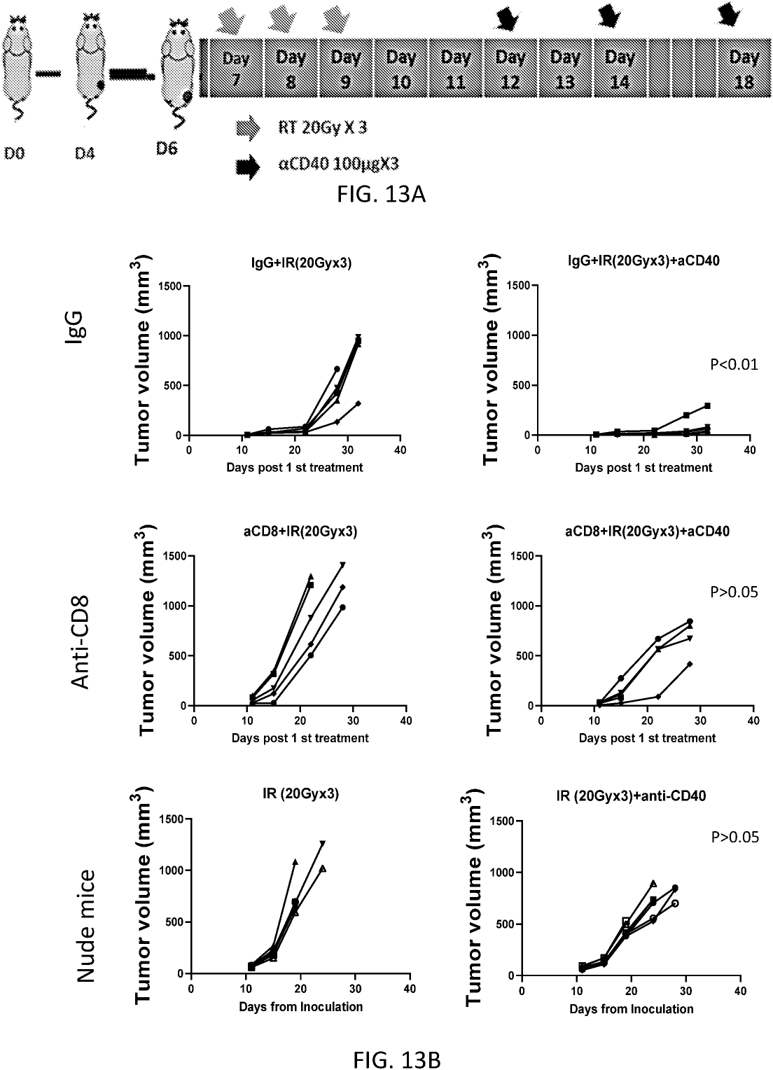

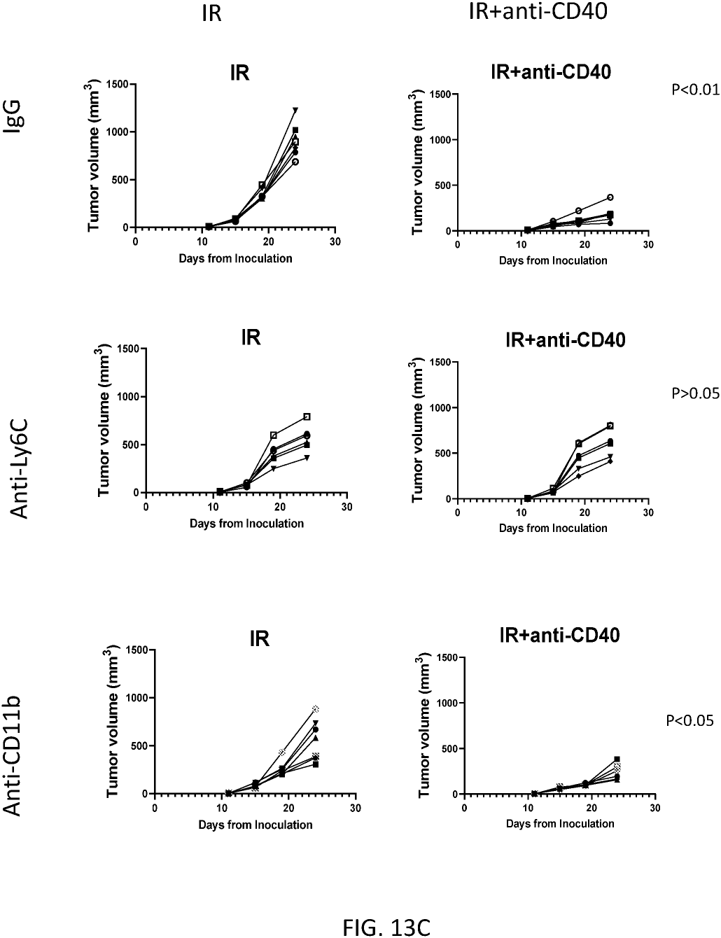

Depletion experiments were performed to investigate the role of different subsets of the immune cells in mediating the therapeutic effect of the anti-CD40 and IR combination. The experimental protocol is depicted in FIG. 13A. anti-CD8, anti-CD11b, anti-LY6C antibodies were injected at D -4 Day 0 and was injected at every 4th day till the termination of the experiment.

For immune-phenotyping studies, on 17th day post tumor inoculation tumors were excised postmortem and dissociated using a cocktail of collagenase type IV and DNase. After digestion at 37° C. for 30 minutes, cells were passed through a 70-µm filter. Cells were stained for cell surface and cytosolic proteins and analyzed by flow cytometry as previously and zombie IR (Thermo Fisher) was used as a viability dye.

To investigate the role of the CD8 T cells in the therapeutic efficacy of IR+ anti-CD40 combination group, anti-CD8 antibodies were used to deplete CD8 cells in the C57BL6 mice. Tumor growth delay in the IR+anti-CD40 combination was partially reversed in the anti-CD8 depleted mice, as depicted in FIG. 13B (middle panels).

Homozygous athymic nude mice lack T cells and suffer from a lack of cell-mediated immunity. Homozygous nude mice also show partial defect in B cell development. Similar results were also observed in the nude mice experiments where the effect of combination was not significant compared to the IR alone group, as depicted in FIG. 13B (lower panels). These results suggested the therapeutic effect of the IR+anti-CD40 combination were mediated partially by CD8 cells.

To further look at which antigen presentation and processing population pool contributed to the therapeutic benefits of the combination group (IR+antiCD40), the LY6C and CD11b population was depleted in the C57BL6 mice. Ly6C high myeloid cells are known to be critical cross presenting APCs along with the dendritic cells. While tumor growth delay observed in the IgG control groups was partially reversed in the CD11b depleted mice, Ly6c depletion completely reversed (p<0.05) the tumor growth delay (FIG. 13C). Ly6C+ myeloid cells have been shown to be to be highly efficient in cross presentation.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A method of treating a tumor or a cancer in an individual, the method comprising administering to the individual a dose of a radiation therapy and a dendritic cell activating molecule, wherein the dendritic cell activating molecule is administered at least one day after the radiation therapy is administered.

2. A method of treating a tumor or a cancer in an individual, the method comprising administering to the individual a dendritic cell activating molecule, wherein the individual has received a dose of a radiation therapy, and wherein the dendritic cell activating molecule is administered at least one day after the radiation therapy has been administered.

3. The method of claim 1 or 2, wherein the dendritic cell activating molecule is administered at least two days after the radiation therapy is administered.

4. The method of claim 1 or 2, wherein the dendritic cell activating molecule is administered at least three days after the radiation therapy is administered.

5. The method of any one of claims 1 or 4, wherein the dose of the radiation therapy comprises a plurality of doses of radiation therapy.

6. The method of any one of claims 1 to 5, wherein the radiation therapy is external beam radiation therapy.

7. The method of any one of claims 1 to 6, wherein the external beam radiation therapy is selected from the list consisting of: three-dimensional conformal radiation therapy, intensity modulated radiation therapy, image guided radiation therapy, stereotactic radiation therapy, intraoperative radiation therapy, proton beam therapy, neutron beam therapy, and combinations thereof.

8. The method of any one of claims 1 to 7, wherein the dose of radiation therapy comprises at least about 2 Gy.

9. The method of any one of claims 1 to 7, wherein the dose of radiation therapy comprises at least about 2 Gy and no more than about 20 Gy.

10. The method of any one of claims 1 to 9, wherein the dendritic cell activating molecule is administered at least three days after the dose of the radiation therapy.

11. The method of any one of claims 1 to 9, wherein the dendritic cell activating molecule is administered at least five days after the dose of the radiation therapy.

12. The method of any one of claims 1 to 9, wherein the dendritic cell activating molecule is administered at least seven days after the dose of the radiation therapy.

13. The method of any one of claims 1 to 12, wherein the dendritic cell activating molecule induces maturation of an immature dendritic cell.

14. The method of any one of claims 1 to 12, wherein the dendritic cell activating molecule activates dendritic cell activation through a toll-like receptor, a NOD-like receptor, a RIG-1 or MDA-5 receptor, a C-type lectin receptor, a costimulatory molecule, a cytokine receptor, or a STING pathway.

15. The method of any one of claims 1 to 12, wherein the dendritic cell activating molecule is a toll-like receptor agonist selected from the list consisting of CpG oligonucleotide, SD-101, LFX453, imiquimod, Bacillus Calmette-Guérin (BCG), monophosphoryl lipid A, Poly ICLC, GSK1795091, and combinations thereof.

16. The method of any one of claims 1 to 12, wherein the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof.

17. The method of any one of claims 1 to 12, wherein the dendritic cell activating molecule is a RIG-1 or MDA-5 receptor agonist selected from the list consisting of poly(I:C), Poly(dA:dT), Poly(dG:dC), 3p-hpRNA, 5′ppp-dsRNA, and combinations thereof.

18. The method of any one of claims 1 to 12, wherein the dendritic cell activating molecule is a C-type lectin receptor agonist selected from the list consisting of Beta-1,3-glucan, zymosan, Heat-killed C. albicans, cord factor, and Trehalose-6,6-dibehenate, and combinations thereof.

19. The method of any one of claims 1 to 12, wherein the dendritic cell activating molecule is a costimulatory molecule agonist selected from the list consisting of a CD40 agonist, aCD80 agonist, a CD86 agonist, an OX40 agonist, and combinations thereof.

20. The method of claim 19, wherein the CD40 agonist is an anti-CD40 agonistic antibody.

21. The method of claim 20, wherein the anti-CD40 agonistic antibody comprises dacetuzumab, CP-870,893, ADC-1013, 2141-v11, APX005M, Chi Lob 7/4, BG9588 (NIAMS), CFZ533, PG10, BMS-986004, lucatumumab, HCD122, JNJ-64457107, selicrelumab, ASKP1240, CDX-1140, or SEA-CD40.

22. The method of any one of claims 1 to 12, wherein the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof.

23. The method of any one of claims 1 to 12, wherein the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB1891, E-7766, or SB11285, and combinations thereof.

24. The method of any one of claims 1 to 23, wherein the dendritic cell activating molecule is administered to a tumor being treated with the dose of the radiation therapy.

25. The method of any one of claims 1 to 24, wherein the tumor or the cancer is a solid tissue tumor or cancer.

26. The method of claim 25, wherein the solid tissue tumor or cancer is of breast, prostate, or a melanoma.

27. The method of any one of claims 1 to 24, wherein the tumor or cancer is resistant to checkpoint inhibitor therapy.

28. The method of claim 27, wherein the checkpoint inhibitor therapy comprises anti-PD1, anti-PDL1, or anti-CTLA4.

29. A method of treating a tumor or a cancer in an individual, the method comprising administering to the individual a dose of an energy-based therapy and a dendritic cell activating molecule, wherein the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy.

30. A method of treating a tumor or a cancer in an individual, the method comprising administering to the individual a dendritic cell activating molecule, wherein the individual has been administered a dose of an energy-based therapy, wherein the dose of the energy-based therapy is selected from the list consisting of Irreversible Electroporation (IRE), Microwave, Low-Intensity Focused Ultrasound (LOFU), High-Intensity Focused Ultrasound (HIFU), Radiofrequency energy, and cryotherapy.

31. The method of claim 29 or 30, wherein the dose of the energy-based therapy comprises a plurality of doses of energy-based therapy.

32. The method of any one of claims 29 to 31, wherein the energy-based therapy is Irreversible Electroporation (IRE).

33. The method of any one of claims 29 to 31, wherein the energy-based therapy is microwave therapy.

34. The method of any one of claims 29 to 31, wherein the energy-based therapy is Low-Intensity Focused Ultrasound (LOFU).

35. The method of claim 34, wherein the LOFU is administered at an intensity of between 10 and 1000 W/cm² in the area of treatment.

36. The method of any one of claims 29 to 31, wherein the energy-based therapy is High-Intensity Focused Ultrasound (HIFU).

37. The method of claim 36, wherein the HIFU is administered at an intensity of between 1,000 and 10,000 W/cm² in the area of treatment.

38. The method of any one of claims 29 to 31, wherein the energy-based therapy is cryotherapy.

39. The method of any one of claims 29 to 38, wherein the dendritic cell activating molecule is administered at least three days after the dose of the energy-based therapy.

40. The method of any one of claims 29 to 38, wherein the dendritic cell activating molecule is administered at least five days after the dose of the energy-based therapy.

41. The method of any one of claims 29 to 38, wherein the dendritic cell activating molecule is administered at least seven days after the dose of the energy-based therapy.

42. The method of any one of claims 29 to 41, wherein the dendritic cell activating molecule activates maturation of an immature dendritic cell.

43. The method of any one of claims 29 to 41, wherein the dendritic cell activating molecule activates dendritic cell activation through a toll-like receptor, a NOD-like receptor, a RIG-1 or MDA-5 receptor, a C-type lectin receptor, a costimulatory molecule, a cytokine receptor, or a STING pathway.

44. The method of any one of claims 29 to 41, wherein the dendritic cell activating molecule is a toll-like receptor agonist selected from the list consisting of CpG oligonucleotide, SD-101, LFX453, imiquimod, Bacillus Calmette-Guérin (BCG), monophosphoryl lipid A, Poly ICLC, GSK1795091, and combinations thereof.

45. The method of any one of claims 29 to 41, wherein the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof.

46. The method of any one of claims 29 to 41, wherein the dendritic cell activating molecule is a RIG-1 or MDA-5 receptor agonist selected from the list consisting of poly(I:C), Poly(dA:dT), Poly(dG:dC), 3p-hpRNA, 5′ppp-dsRNA, and combinations thereof.

47. The method of any one of claims 29 to 41, wherein the dendritic cell activating molecule is a C-type lectin receptor agonist selected from the list consisting of Beta-1,3-glucan, zymosan, Heat-killed C. albicans, cord factor, and Trehalose-6,6-dibehenate, and combinations thereof.

48. The method of any one of claims 29 to 41, wherein the dendritic cell activating molecule is a costimulatory molecule agonist selected from the list consisting of a CD40 agonist, aCD80 agonist, a CD86 agonist, an OX40 agonist, and combinations thereof.

49. The method of claim 48, wherein the CD40 agonist is an anti-CD40 agonistic antibody.

50. The method of claim 49, wherein the anti-CD40 agonistic antibody comprises dacetuzumab, CP-870,893, ADC-1013, 2141-v11, APX005M, Chi Lob 7/4, BG9588 (NIAMS), CFZ533, PG10, BMS-986004, lucatumumab, HCD122, JNJ-64457107, selicrelumab, ASKP1240, CDX-1140, or SEA-CD40.

51. The method of any one of claims 29 to 41, wherein the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof.

52. The method of any one of claims 29 to 41, wherein the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB1891, E-7766, or SB11285, and combinations thereof.

53. The method of any one of claims 29 to 52, wherein the dendritic cell activating molecule is administered to a tumor being treated with the dose of the energy-based therapy.

54. The method of any one of claims 29 to 52, wherein the tumor or the cancer is a solid tissue tumor or cancer.

55. The method of claim 54, wherein the solid tissue tumor or cancer is of breast, prostate, or a melanoma.

56. The method of any one of claims 29 to 52, wherein the tumor or cancer is resistant to checkpoint inhibitor therapy.

57. The method of claim 56, wherein the checkpoint inhibitor therapy comprises anti-PD1, anti-PDL1, or anti-CTLA4.

58. A method of increasing T cell infiltration into a tumor distal to a tumor being treated in an individual, the method comprising administering to the individual a dose of a radiation therapy and a dendritic cell activating molecule, wherein the dendritic cell activating molecule is administered at least one day after the radiation therapy is administered.

59. The method of claim 58, wherein the dendritic cell activating molecule is administered at least two days after the radiation therapy is administered.

60. The method of claim 58, wherein the dendritic cell activating molecule is administered at least three days after the radiation therapy is administered.

61. The method of any one of claims 58 to 60, wherein the dose of the radiation therapy comprises a plurality of doses of radiation therapy.

62. The method of any one of claims 58 to 61, wherein the radiation therapy is external beam radiation therapy.

63. The method of any one of claims 58 to 62, wherein the external beam radiation therapy is selected from the list consisting of: three-dimensional conformal radiation therapy, intensity modulated radiation therapy, image guided radiation therapy, stereotactic radiation therapy, intraoperative radiation therapy, proton beam therapy, neutron beam therapy, and combinations thereof.

64. The method of any one of claims 58 to 62, wherein the dose of radiation therapy comprises at least about 2 Gy.

65. The method of any one of claims 58 to 62, wherein the dose of radiation therapy comprises at least about 2 Gy and no more than about 20 Gy.

66. The method of any one of claims 58 to 65, wherein the dendritic cell activating molecule is administered at least three days after the dose of the radiation therapy.

67. The method of any one of claims 58 to 65, wherein the dendritic cell activating molecule is administered at least five days after the dose of the radiation therapy.

68. The method of any one of claims 58 to 65, wherein the dendritic cell activating molecule is administered at least seven days after the dose of the radiation therapy.

69. The method of any one of claims 58 to 68, wherein the dendritic cell activating molecule activates maturation of an immature dendritic cell.

70. The method of any one of claims 58 to 68, wherein the dendritic cell activating molecule activates dendritic cell activation through a toll-like receptor, a NOD-like receptor, a RIG-1 or MDA-5 receptor, a C-type lectin receptor, a costimulatory molecule, a cytokine receptor, or a STING pathway.

71. The method of any one of claims 58 to 68, wherein the dendritic cell activating molecule is a toll-like receptor agonist selected from the list consisting of CpG oligonucleotide, SD-101, LFX453, imiquimod, Bacillus Calmette-Guérin (BCG), monophosphoryl lipid A, Poly ICLC, GSK1795091, and combinations thereof.

72. The method of any one of claims 58 to 69, wherein the dendritic cell activating molecule is a NOD-like receptor agonist selected from the list consisting of bacterial peptidoglycan, an acylated derivative of iE-DAP (C12-iE-DAP), D-gamma-Glu-mDAP (iE-DAP), L-Ala-gamma-D-Glu-mDAP (Tri-DAP), muramyl dipeptide (MDP), muramyl tripeptide, L18-MDP, M-TriDAP, murabutide, PGN-ECndi, PGN-ECndss, PGN-SAndi, N-glycolylated muramyl dipeptide, murabutide, and combinations thereof.

73. The method of any one of claims 58 to 69, wherein the dendritic cell activating molecule is a RIG-1 or MDA-5 receptor agonist selected from the list consisting of poly(I:C), Poly(dA:dT), Poly(dG:dC), 3p-hpRNA, 5′ppp-dsRNA, and combinations thereof.

74. The method of any one of claims 58 to 69, wherein the dendritic cell activating molecule is a C-type lectin receptor agonist selected from the list consisting of Beta-1,3-glucan, zymosan, Heat-killed C. albicans, cord factor, and Trehalose-6,6-dibehenate, and combinations thereof.

75. The method of any one of claims 58 to 69, wherein the dendritic cell activating molecule is a costimulatory molecule agonist selected from the list consisting of a CD40 agonist, aCD80 agonist, a CD86 agonist, an OX40 agonist, and combinations thereof.

76. The method of claim 75, wherein the CD40 agonist is an anti-CD40 agonistic antibody.

77. The method of claim 76, wherein the anti-CD40 agonistic antibody comprises dacetuzumab, CP-870,893, ADC-1013, 2141-v11, APX005M, Chi Lob 7/4, BG9588 (NIAMS), CFZ533, PG10, BMS-986004, lucatumumab, HCD122, JNJ-64457107, selicrelumab, ASKP1240, or SEA-CD40.

78. The method of any one of claims 58 to 69, wherein the dendritic cell activating molecule is a cytokine selected from the list consisting of granulocyte macrophage colony stimulating factor (GM-CSF), interleukin-15 (IL-15), tumor necrosis factor alpha (TNF-alpha), interferon gamma (IFN-gamma), and combinations thereof.

79. The method of any one of claims 58 to 69, wherein the dendritic cell activating molecule is a STING agonist selected from the list consisting of 2′,3′-cGAMP (CAS Number, 1441190-66-4), 4-[(2-Chloro-6-fluorophenyl)methyl]-N-(furan-2-ylmethyl)-3-oxo-1,4-benzothiazine-6-carboxamide, MK-1454, ADU-S100/MIW815, SRCB-0074, SYNB1891, E-7766, or SB11285, and combinations thereof.

80. The method of any one of claims 58 to 79, wherein the dendritic cell activating molecule is administered to a tumor being treated with the dose of the radiation therapy.

81. The method of any one of claims 58 to 80, wherein the tumor or the cancer is a solid tissue tumor or cancer.

82. The method of claim 81, wherein the solid tissue tumor or cancer is of breast, prostate, or a melanoma.

83. The method of any one of claims 58 to 80, wherein the tumor or cancer is resistant to checkpoint inhibitor therapy.

84. The method of claim 83, wherein the checkpoint inhibitor therapy comprises anti-PD1, anti-PDL1, or anti-CTLA4.
